(19) United States
(12) Patent Application Publication
(10) Pub. No.: US 2014/0106985 A1
(43) Pub. Date: Apr. 17, 2014

(54) MICRORNA BIOMARKERS FOR PROGNOSIS OF PATIENTS WITH PANCREATIC CANCER

(71) Applicants: Julia Sidenius Johansen, Frederiksberg (DK); Nicolai Aagaard Schultz, Frederiksberg C (DK); Morten Wojdemann, Valby (DK)

(72) Inventors: Julia Sidenius Johansen, Frederiksberg (DK); Nicolai Aagaard Schultz, Frederiksberg C (DK); Morten Wojdemann, Valby (DK)

(73) Assignee: HERLEV HOSPITAL, Herlev (DK)

(21) Appl. No.: 14/117,975

(22) PCT Filed: May 16, 2012
(86) PCT No.: PCT/DK2012/050172

§ 371 Date: Nov. 15, 2013

Related U.S. Application Data
(60) Provisional application No. 61/486,827, filed on May 17, 2011.

(30) Foreign Application Priority Data
May 17, 2011 (DK) PA201170243

(51) Int. Cl. C12Q 1/68

(52) U.S. Cl. CPC C12Q 1/6886
USPC 506/9; 435/6.12; 536/24.31; 506/16; 435/287.2; 435/6.11

(57) ABSTRACT

The present invention relates to prognostic micro RNA (miRNA) biomarkers based on a specific miRNA expression pattern, which can prove as a valuable prognostic tool to predict the survival of patients being diagnosed with pancreas cancer.

All patent and non-patent references cited in the application are hereby incorporated by reference in their entirety.

### FIELD OF INVENTION

The present invention relates to a means for improving the prognosis of patients with pancreatic cancer. Prognostic microRNA (miRNA) biomarkers and classifiers based on a specific miRNA expression pattern are disclosed herein, which can prove as a valuable prognostic tool to make possible to predict the survival of patients being diagnosed with pancreas cancer.

### BACKGROUND OF INVENTION

Pancreatic cancer (PC) is the 4^{th} most common cause of cancer death in United States and Europe. The prognosis of patients with pancreatic cancer is dismal with a 5-year survival rate of less than 5% and a median survival from diagnosis around 3 to 6 months. Complete remission is rare.

The poor prognosis is partly because the cancer usually causes no symptoms early on, leading to locally advanced or metastatic pancreatic cancer at the time of diagnosis. Fewer than 10% of patients' tumours are confined to the pancreas at the time of diagnosis. In most cases, the malignancy has already progressed to the point where surgical removal is impossible.

In those cases where resection can be performed, the average survival rate increases to 18 to 20 months. The overall five-year survival rate is about 10%, although this can rise as high as 20% to 25% if the tumour is removed completely and when cancer has not spread to lymph nodes.

MicroRNAs (miRNA or miR) are small, non-coding single-stranded RNA gene products that regulate mRNA translation. The expression of RNA species, such as miRNAs is often deregulated in malignant cells and shows a highly tissue-specific pattern. miRNA biomarkers whose expression is associated with a certain condition, and classifiers based on a miRNA expression profile or signature, may prove to be an ideal prognostic tool to evaluate the prognosis of individual patients with pancreatic cancer.

It has been demonstrated that pancreatic cancer has a miRNA expression pattern that differs from normal pancreas and chronic pancreatitis tissue, and that may be correlated to the prognostic outcome for a patient with pancreatic cancer (see e.g. Mardin & Mees, Ann. Surg. Oncol. (2009) 16:3183-3189).

Mardin & Mees reviews the results from a range of scientific groups with respect to differential expression of specific miRNAs in pancreas cancer versus normal pancreas. These miRs are however not associated directly with the ability to predict the prognosis or survival for a patient with pancreatic cancer—in fact, there may be no association, but they may potentially be used to distinguish normal and cancerous tissues. Amongst others, miR-21, miR-196a-2 and miR-187 are identified as miRs with prognostic potential in the Mardin and Mees review.

Greither et al. (Int. J. Cancer 2009, DOI 10.1002/ijc.24687) show that elevated expression of miR-155, 203, 210 and 222 in pancreas tumours are associated with poorer survival, whereas miR-216 and 217 had no association despite their previous identification as deregulated in pancreas cancer.

While the literature has addressed the miRNA expression pattern in pancreas cancer versus normal tissue, and some have further associated certain miRNAs with survival and prognostic outcome, the present inventors aimed to improve and further develop prognostic tools for providing a more accurate prognosis of patients with pancreatic cancer.

### SUMMARY OF INVENTION

Efforts to make possible an improved prognosis for pancreas cancer patients are needed, in order to improve and specify the individual follow-up therapy after surgery and diagnosis and management after diagnosis of the condition for each patient with pancreatic cancer.

The present inventors have further investigated the miRNA expression profile in pancreatic cancer (PC) samples obtained from PC patients (comprising pancreatic adenocarcinoma; PAC and ampullary adenocarcinoma, AAC), in order to identify specific miRNAs associated with the prognosis of said PC patients.

This has lead to the identification of a subset of miRNAs associated with the survival of said PC patients; overall survival (OS) and 2-year follow up, including miRNAs which have not previously been identified by others. These miRNAs are potentially useful in assessing or determining the prognosis of an individual patient with pancreatic cancer.

The present invention thus discloses a means of assessing, determining or predicting the prognosis of a patient with pancreatic cancer. The inventors have found that a subset of specific miRNAs are differentially expressed in and associated with the OS and/or 2-years follow-up survival of pancreas cancer patients.

By employing the prognostic miRNA biomarkers (alone or in ‘simple combinations’) disclosed herein, it is thus made possible to predict the prognosis of a diseased individual suffering from pancreatic cancer. The quality of said prediction is at least comparable to other prognostic biomarkers for PC, and in some embodiments yields an improved prognosis as compared to those provided thus far.

The present invention is in one aspect directed to the identification of prognostic miRNA biomarkers whose expression level is associated with estimating the prognosis of PC patients.

Accordingly, provided herein are methods for predicting the prognosis for a patient with pancreatic cancer, said method comprising measuring the expression level of at least one miRNA in a sample obtained from said individual, determining whether or not said sample is indicative of the individual having a certain predicted prognosis.

Said method may be a method for estimating the probability for a patient with pancreatic cancer of surviving for a certain time period.

The at least one miRNAs according to the present invention are selected from the group consisting of miR-675, miR-212, miR-148a*, miR-187, let-7g*, miR-205, miR-944, miR-431, miR-194*, miR-769-5p, miR-450b-5p, miR-222, miR-146a, miR-23a*, miR-143*, miR-216a, miR-891a, miR-409-5p, miR-449b and miR-330-5p.

In one embodiment, one or more miRNAs according to the present invention are selected from the group consisting of miR-675, miR-212, miR-148a*, miR-187 and let-7g*; or the group consisting of miR-675, miR-212, miR-148a*, miR-187, miR-205, miR-944, miR-431, miR-194* and miR-769-5p; or the group consisting of miR-675, miR-212, miR-148a* and miR-187; form part of the present invention.

In another embodiment, one or more miRNAs according to the present invention are selected from the group consisting of miR-675, miR-148a*, miR-450b-5p, miR-222 and miR-146a; or miR-675, miR-148a*, miR-187, miR-205, miR-431, miR-194*, miR-23a*, miR-143*, miR-216a, miR-891a, miR-409-5p, miR-450b-5p, miR-449b and miR-330-5p; or miR-675, miR-148a* and miR-450b-5p; form part of the present invention.

The miRNA biomarkers may be applied ex vivo to a sample obtained from an individual, in order to facilitate an improved prognosis of said individual. Said sample may be a tissue sample from the pancreas obtained from an individual.

The use of the herein disclosed miRNA biomarkers can potentially improve the prognosis of pancreas cancer patients, and is as such useful as a stand-alone or an ‘add-on’ method to the existing prognostic means currently used for estimating the prognosis of a pancreas cancer patient.

The present invention is also directed to a device comprising probes for at least one miRNA according to the present invention; suitable for measuring the expression level of said at least one miRNA, wherein said device may be used for estimating the prognosis of a pancreas cancer patient.

Also provided is a system for predicting the prognosis for a patient with pancreatic cancer, comprising means for analysing the expression level of at least one miRNA in a sample obtained from an individual with pancreas cancer, and means for determining the prognosis for said individual.

The present invention is also directed to a computer program product having a computer readable medium, said computer program product providing a system for predicting the prognosis of an individual, said computer program product comprising means for carrying out any of the steps of any of the methods as disclosed herein.

### DESCRIPTION OF THE DRAWINGS

FIG. 1: CONSORT diagram.

FIG. 2: Kaplan-Meier survival curves for microRNAs associated with OS (A-D). Blue line corresponds to a 10 percentile microRNA expression in PC tissue and red line corresponds to a 90 percentile microRNA expression in PC tissue.

FIG. 3: Nomogram for the multivariate survival model predicting OS in patients operated for PC. To estimate risk, calculate points for each microRNA expression in PC tissue by drawing a straight line from patient's microRNA to the axis labeled “points”. Sum all points and draw a straight line from the total point axis to 3-months, 1-year and 10-year survival probability axes.

FIG. 4: Kaplan-Meier curves for groups above (red) and below (blue) median prognostic index (PI).

FIG. 5: Kaplan-Meier survival curves for microRNAs significant for OS in the CPH-model with only 2-years follow-up (A-E). Blue line corresponds to a 10 percentile microRNA expression in PC tissue and red line corresponds to a 90 percentile microRNA expression in PC tissue.

### DEFINITIONS

Prognosis: A prediction of the probable course and outcome of a disease; the likelihood of recovery from a disease. A forecast or prediction.

Overall survival (OS): The outcome for the patients included in a study from the point of inclusion (surgery) until their death or emigration/censoring.

‘2-years follow up’ survival: The outcome for the patients included in a study from the point of inclusion (surgery) until 2 years after inclusion. Survival times above 2 years are censored in the analysis.

Statistical classification is a procedure in which individual items are placed into groups based on quantitative information on one or more characteristics inherent in the items (referred to as traits, variables, characters, etc) and based on a training set of previously labeled items.

A classifier is a prediction model which may distinguish between or characterize samples by classifying a given sample into a predetermined class based on certain characteristics of said sample. A two-way classifier classifies a given sample into one of two predetermined classes, and a three-way classifier classifies a given sample into one of three predetermined classes.

The terms distinction, differentiation, separation, classification and characterisation of a sample are used herein as being capable of predicting with a relatively high sensitivity and specificity if a given sample of unknown prognosis belongs to the class of one of two given classes; each class representing a predicted estimated survival. The output may be given as a probability of belonging to either class of between 0-1 (for classifiers), it may be given as a probability of prediction probability for a certain survival (for biomarkers, or may be estimated directly based on differences in expression levels (for biomarkers).

A tiomarker may be defined as a biological molecule found in blood, other body fluids, or tissues that is an indicator of a normal or abnormal process, or of a condition or disease. A biomarker may be used to foresee how well the body responds to a treatment for a disease or condition, or may be used to associate a certain disease or condition—or outcome of disease—to a certain value of said biomarker found in e.g. a tissue sample. Biomarkers are also called molecular marker and signature molecule. If the biomarker is used to predict the probable course and outcome of a disease, it may be called a prognostic biomarker.

‘Collection media’ as used herein denotes any solution suitable for collecting, storing or extracting of a sample for immediate or later retrieval of RNA from said sample.

‘Deregulated’ means that the expression of a gene or a gene product is altered from its normal baseline levels; comprising both up- and down-regulated.

The term “Individual” refers to vertebrates, particular members of the mammalian species, preferably primates including humans. As used herein, ‘subject’ and ‘individual’ may be used interchangeably.

The term “Kit of parts” as used herein provides a device for measuring the expression level of at least one miRNA as identified herein, and at least one additional component. The additional component may be used simultaneously, sequentially or separately with the device. The additional component may in one embodiment be means for extracting RNA, such as miRNA, from a sample; reagents for performing microarray analysis, reagents for performing QPCR analysis and/or instructions for use of the device and/or additional components.

The term “natural nucleotide” or “nucleotide” refers to any of the four deoxyribonucleotides, dA, dG, dT, and dC (constituents of DNA), and the four ribonucleotides, A, G, U, and C (constituents of RNA). Each natural nucleotide comprises or essentially consists of a sugar moiety (ribose or deoxyribose), a phosphate moiety, and a natural/standard base moiety. Natural nucleotides bind to complementary nucleotides according to well-known rules of base pairing (VVatson and Crick), where adenine (A) pairs with thymine (T) or uracil (U); and where guanine (G) pairs with cytosine (C), wherein corresponding base-pairs are part of complementary, anti-parallel nucleotide strands. The base pairing results in a specific hybridization between predetermined and complementary nucleotides. The base pairing is the basis by which enzymes are able to catalyze the synthesis of an oligonucleotide complementary to the template oligonucleotide. In this synthesis, building blocks (normally the triphosphates of ribo or deoxyribo derivatives of A, T, U, C, or G) are directed by a template oligonucleotide to form a complementary oligonucleotide with the correct, complementary sequence. The recognition of an oligonucleotide sequence by its complementary sequence is mediated by corresponding and interacting bases forming base pairs. In nature, the specific interactions leading to base pairing are governed by the size of the bases and the pattern of hydrogen bond donors and acceptors of the bases. A large purine base (A or G) pairs with a small pyrimidine base (T, U or C). Additionally, base pair recognition between bases is influenced by hydrogen bonds formed between the bases. In the geometry of the Watson-Crick base pair, a six membered ring (a pyrimidine in natural oligonucleotides) is juxtaposed to a ring system composed of a fused, six membered ring and a five membered ring (a purine in natural oligonucleotides), with a middle hydrogen bond linking two ring atoms, and hydrogen bonds on either side joining functional groups appended to each of the rings, with donor groups paired with acceptor groups.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g. alpha-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes e.g. so-called “peptide nucleic acids,” which comprise naturally-occurring or modified nucleic acid bases attached to a polyamide backbone. Nucleic acids can be either single stranded or double stranded. In an aspect of the present invention, ‘nucleic acid’ is meant to comprise antisense oligonucleotides (ASO), small inhibitory RNAs (sRNA), short hairpin RNA (shRNA) and microRNA (miRNA).

A “polypeptide” or “protein” is a polymer of amino acid residues preferably joined exclusively by peptide bonds, whether produced naturally or synthetically. The term “polypeptide” as used herein covers proteins, peptides and polypeptides, wherein said proteins, peptides or polypeptides may or may not have been post-translationally modified. Post-translational modification may for example be phosphorylation, methylation and glycosylation.

A ‘probe’ as used herein refers to a hybridization probe. A hybridization probe is a (single-stranded) fragment of DNA or RNA of variable length (usually 100-1000 bases long), which is used in DNA or RNA samples to detect the presence of nucleotide sequences (the DNA target) that are complementary to the sequence in the probe. The probe thereby hybridizes to single-stranded nucleic acid (DNA or RNA) whose base sequence allows probe-target base pairing due to complementarity between the probe and target. To detect hybridization of the probe to its target sequence, the probe is tagged (or labelled) with a molecular marker of either radioactive or fluorescent molecules. DNA sequences or RNA transcripts that have moderate to high sequence similarity to the probe are then detected by visualizing the hybridized probe. Hybridization probes used in DNA microarrays refer to DNA covalently attached to an inert surface, such as coated glass slides or gene chips, and to which a mobile cDNA target is hybridized.

Due to the imprecision of standard analytical methods, molecular weights and lengths of polymers are understood to be approximate values. When such a value is expressed as “about” X or “approximately” X, the stated value of X will be understood to be accurate to +/−20%, such as +/−10%, for example +/−5%.

### DETAILED DESCRIPTION OF THE INVENTION

### The Pancreas

The pancreas is a gland organ in the digestive and endocrine system of vertebrates. It is both an endocrine gland producing several important hormones, including insulin, glucagon, and somatostatin, as well as an exocrine gland, secreting pancreatic juice containing digestive enzymes that pass to the small intestine. These enzymes help to further break down the carbohydrates, proteins, and fats in the chyme.

Microscopically, stained sections of the pancreas reveal two different types of parenchymal tissue. Lightly staining clusters of cells are called islets of Langerhans, which produce hormones that underlie the endocrine functions of the pancreas. Darker staining cells form acini connected to ducts. Acinar cells belong to the exocrine pancreas and secrete digestive enzymes into the gut via a system of ducts.

Four main cell types exist in the islets of Langerhans that can be classified by their secretion: a (alpha) cells secrete glucagon (increase glucose in blood), α (beta) cells secrete insulin (decrease glucose in blood), δ (delta) cells secrete somatostatin (regulates α and β cells), and PP cells secrete pancreatic polypeptide.

The pancreas receives regulatory innervation via hormones in the blood and through the autonomic nervous system. These two inputs regulate the secretory activity of the pancreas.

The pancreas lies in the epigastrium and left hypochondrium areas of the abdomen. The head lies within the concavity of the duodenum. The uncinate process emerges from the lower part of head, and lies deep to superior mesenteric vessels. The neck is the constricted part between the head and the body. The body lies behind the stomach. The tail is the left end of the pancreas. It lies in contact with the spleen and runs in the lienorenal ligament.

### Pancreatic Cancer

Neoplasia or cancer is the abnormal proliferation of cells, resulting in a structure known as a neoplasm. The growth of this clone of cells exceeds, and is uncoordinated with, that of the normal tissues around it. It usually causes a lump or tumour. Neoplasias may be benign (adenoma) or malignant (carcinoma).

Pancreatic or pancreas neoplasia, pancreatic or pancreas cancer (PC), pancreatic or pancreas carcinoma may be used interchangeably throughout the present application. Normal pancreas is abbreviated NP.

Pancreatic cancer is a malignant neoplasm of the pancreas. Patients diagnosed with pancreatic cancer have a poor prognosis, partly because the cancer usually causes no symptoms early on, leading to locally advanced or metastatic disease at the time of diagnosis. Median survival from diagnosis is around 3 to 6 months; 5-year survival is less than 5%. Pancreatic cancer has one of the highest fatality rates of all cancers, and is the fourth-highest cancer killer in the US and Europe.

The vast majority; about 95% of exocrine pancreatic cancers are pancreatic adenocarcinomas; PAC (also known as pancreatic ductal adenocarcinoma, PDAC). Accordingly, PC and PAC are often used as synonyms. The remaining 5% include adenosquamous carcinomas, signet ring cell carcinomas, hepatoid carcinomas, colloid carcinomas, undifferentiated carcinomas, and undifferentiated carcinomas with osteoclast-like giant cells. Exocrine pancreatic tumours are far more common than pancreatic endocrine tumours, which make up about 1% of total cases.

Desmoplasia is the growth of fibrous or connective tissue. It is also called desmoplastic reaction to emphasize that it is secondary to a neoplasm, causing dense fibrosis around the tumour. Desmoplasia is usually only associated with malignant neoplasms, such as pancreas cancer which can evoke a fibrosis response by invading healthy tissue.

Treatment of pancreatic cancer depends on the stage of the cancer. The Whipple procedure is the most common surgical treatment for cancers involving the head of the pancreas. This procedure involves removing the pancreatic head and the curve of the duodenum together (pancreato-duodenectomy), making a bypass for food from stomach to jejunum (gastro-jejunostomy) and attaching a loop of jejunum to the cystic duct to drain bile (cholecysto-jejunostomy). It can be performed only if the patient is likely to survive major surgery and if the cancer is localized without invading local structures or metastasizing. It can, therefore, be performed in only the minority of patients.

Cancers of the tail of the pancreas can be resected using a procedure known as a distal pancreatectomy. Recently, localized cancers of the pancreas have been resected using minimally invasive (laparoscopic) approaches.

Surgery can be performed for palliation, if the malignancy is invading or compressing the duodenum or colon. In that case, bypass surgery might overcome the obstruction and improve quality of life, but it is not intended as a cure.

After surgery, adjuvant chemotherapy has been shown to significantly increase the 5-year survival, and should be offered if the patient is fit after surgery. Addition of radiation therapy is a debated topic, due to the lack of any large randomized studies to show any survival benefit of this strategy.

In patients not suitable for resection with curative intent, palliative chemotherapy may be used to improve quality of life and gain a modest survival benefit.

### Ampullary Adenocarcinoma

Ampullary adenocarcinomas (A-AC or AAC); also known as adenocarcinoma of the Ampulla of Vater, is a malignant tumour arising in the last centimeter of the common bile duct, where it passes through the wall of the duodenum and ampullary papilla. The pancreatic duct (of Wirsung) and common bile duct merge and exit by way of the ampulla into the duodenum. The ductal epithelium in these areas is columnar and resembles that of the lower common bile duct.

AAC is relatively uncommon, accounting for approximately 0.2% of gastrointestinal tract malignancies and approximately 7% of all periampullary carcinomas.

The prognosis of AAC is better than for PAC with a 5-years survival after surgery of 40%. One of the reasons is that even small AAC cause jaundice so more patients are operated at an early tumour stage and without lymph node metastasis.

### Symptoms and Diagnosis

Pancreatic cancer is sometimes called a “silent killer” because early pancreatic cancer often does not cause symptoms, and the later symptoms are usually nonspecific and varied. Therefore, pancreatic cancer is often not diagnosed until it is advanced; hence the poor survival rate.

The clinical and histological similarity between pancreatic cancer and chronic pancreatitis adds another dimension to the diagnostic challenge.

Common symptoms of PC include pain in the upper abdomen that typically radiates to the back, loss of appetite and/or nausea and vomiting, weight loss, painless jaundice, pale-colored stool and steatorrhea, Trousseau sign, diabetes mellitus, or elevated blood sugar levels.

The initial presentation varies according to location of the cancer. Malignancies in the pancreatic body or tail usually present with pain and weight loss, while those in the head of the gland typically present with steatorrhea, weight loss, and jaundice. The recent onset of atypical diabetes mellitus, a history of recent but unexplained thrombophlebitis (Trousseau sign), or a previous attack of pancreatitis are sometimes noted. Courvoisier sign defines the presence of jaundice and a painlessly distended gallbladder as strongly indicative of pancreatic cancer, and may be used to distinguish pancreatic cancer from gallstones. Tiredness, irritability and difficulty eating because of pain also exist. Pancreatic cancer is often discovered during the course of the evaluation of aforementioned symptoms.

Liver function tests can show a combination of results indicative of bile duct obstruction (raised conjugated bilirubin, γ-glutamyl transpeptidase and alkaline phosphatase levels). Imaging studies, such as computed tomography (CT scan) and endoscopic ultrasound (EUS) can be used to identify the location and form of the cancer.

An assessment of risk factors may also help make a diagnosis, comprising the occurrence of pancreatic cancer in the family, age above 60 years, male gender, smoking, obesity, diabetes mellitus, chronic pancreatitis, Helicobacter pylori infection, gingivitis or periodontal disease, diets low in vegetables and fruits, high in red meat, and/or high in sugar-sweetened drinks.

A definitive diagnosis is made by an endoscopic needle biopsy or surgical excision of the radiologically suspicious tissue. Endoscopic ultrasound is often used to visually guide the needle biopsy procedure.

The most common form of pancreatic cancer (ductal adenocarcinoma) is typically characterized by moderately to poorly differentiated glandular structures on microscopic examination. Pancreatic cancer has an immunohistochemical profile that is similar to hepatobiliary cancers (e.g. cholangiocarcinoma) and some stomach cancers; thus, it may not always be possible to be certain that a tumour found in the pancreas arose from it.

Novel strategies for early diagnosis of patients with pancreatic cancer are urgently needed. The use of miRNA expression levels as biomarkers in blood or tissue samples is an emerging research field aimed to improve the diagnostic tools for pancreas cancer. Reference is made to co-pending patent application filed by the overlapping inventors entitled “miRNA for diagnosis of pancreatic cancer” (PA 2010 70574 filed on 22 Dec. 2010)

### Nucleic Acids

A nucleic acid is a biopolymeric macromolecule composed of chains of monomeric nucleotides. In biochemistry these molecules carry genetic information or form structures within cells. The most common nucleic acids are deoxyribonucleic acid (DNA) and ribonucleic acid (RNA). Each nucleotide consists of three components: a nitrogenous heterocyclic base (the nucleobase component), which is either a purine or a pyrimidine; a pentose sugar (backbone residues); and a phosphate group (internucleoside linkers). A nucleoside consists of a nucleobase (often simply referred to as a base) and a sugar residue in the absence of a phosphate linker. Nucleic acid types differ in the structure of the sugar in their nucleotides—DNA contains 2-deoxyriboses while RNA contains ribose (where the only difference is the presence of a hydroxyl group). Also, the nitrogenous bases found in the two nucleic acid types are different: adenine, cytosine, and guanine are found in both RNA and DNA, while thymine only occurs in DNA and uracil only occurs in RNA. Other rare nucleic acid bases can occur, for example inosine in strands of mature transfer RNA. Nucleobases are complementary, and when forming base pairs, must always join accordingly: cytosine-guanine, adenine-thymine (adenine-uracil when RNA). The strength of the interaction between cytosine and guanine is stronger than between adenine and thymine because the former pair has three hydrogen bonds joining them while the latter pair has only two. Thus, the higher the GC content of double-stranded DNA, the more stable the molecule and the higher the melting temperature.

Nucleic acids are usually either single-stranded or double-stranded, though structures with three or more strands can form. A double-stranded nucleic acid consists of two single-stranded nucleic acids held together by hydrogen bonds, such as in the DNA double helix. In contrast, RNA is usually single-stranded, but any given strand may fold back upon itself to form secondary structure as in tRNA and rRNA.

The sugars and phosphates in nucleic acids are connected to each other in an alternating chain, linked by shared oxygens, forming a phosphodiester bond. In conventional nomenclature, the carbons to which the phosphate groups attach are the 3′ end and the 5′ end carbons of the sugar. This gives nucleic acids polarity. The bases extend from a glycosidic linkage to the 1′ carbon of the pentose sugar ring. Bases are joined through N-1 of pyrimidines and N-9 of purines to 1′ carbon of ribose through N-13 glycosyl bond.

### MicroRNA

MicroRNAs (miRNA or miR) are single-stranded RNA molecules of about 19-25 nucleotides in length, which regulate gene expression. MiRNAs are either expressed from non-protein-coding transcripts or mostly expressed from protein coding transcripts. They are processed from primary transcripts known as pri-miRNA to shorter stem-loop structures called pre-miRNA and finally to functional mature miRNA. Mature miRNA molecules are partially complementary to one or more messenger RNA (mRNA) molecules, and their main function is to inhibit gene expression. This may occur by preventing mRNA translation or increasing mRNA turnover/degradation.

The transcripts encoding miRNAs are much longer than the processed mature miRNA molecule; miRNAs are first transcribed as primary transcripts or pri-miRNA with a cap and poly-A tail by RNA polymerase II and processed to short, 70-nucleotide stem-loop structures known as pre-miRNA in the cell nucleus. This processing is performed in animals (including humans) by a protein complex known as the Microprocessor complex, consisting of the ribonuclease III Drosha and the double-stranded RNA binding protein Pasha. These pre-miRNAs are then exported to the cytoplasm by Exportin-5/Ran-GTP and processed to mature miRNAs by interaction with the ribonuclease III Dicer and separation of the miRNA duplexes. The mature single-stranded miRNA is incorporated into a RNA-induced silencing complex (RISC)-like ribonucleoprotein particle (miRNP). The RISC complex is responsible for the gene silencing observed due to miRNA expression and RNA interference. The pathway is different for miRNAs derived from intronic stem-loops; these are processed by Dicer but not by Drosha.

When Dicer cleaves the pre-miRNA stem-loop, two complementary short RNA molecules are formed, but only one is integrated into the RISC complex. This strand is known as the guide strand and is selected by the argonaute protein, the catalytically active RNase in the RISC complex, on the basis of the stability of the 5′ end. The remaining strand, known as the anti-guide or passenger strand, is degraded as a RISC complex substrate. After integration into the active RISC complex, miRNAs base pair with their complementary mRNA molecules. This may induce mRNA degradation by argonaute proteins, the catalytically active members of the RISC complex, or it may inhibit mRNA translation into proteins without mRNA degradation.

The function of miRNAs appears to be mainly in gene regulation. For that purpose, an miRNA is (partly) complementary to a part of one or more mRNAs. Animal (including human) miRNAs are usually complementary to a site in the 3′ UTR. The annealing of the miRNA to the mRNA then inhibits protein translation, and sometimes facilitates cleavage of the mRNA (depending on the degree of complementarity). In such cases, the formation of the double-stranded RNA through the binding of the miRNA to mRNA inhibits the mRNA transcript through a process similar to RNA interference (RNAi). Further, miRNAs may regulate gene expression post-transcriptionally at the level of translational inhibition at P-bodies. These are regions within the cytoplasm consisting of many enzymes involved in mRNA turnover; P bodies are likely the site of miRNA action, as miRNA-targeted mRNAs are recruited to P bodies and degraded or sequestered from the translational machinery. In other cases it is believed that the miRNA complex blocks the protein translation machinery or otherwise prevents protein translation without causing the mRNA to be degraded. miRNAs may also target methylation of genomic sites which correspond to targeted mRNAs. miRNAs function in association with a complement of proteins collectively termed the miRNP (miRNA ribonucleoprotein complex).

Under a standard nomenclature system, miRNA names are assigned to experimentally confirmed miRNAs before publication of their discovery. The prefix “mir” is followed by a dash and a number, the latter often indicating order of naming. For example, mir-123 was named and likely discovered prior to mir-456. The uncapitalized “mir-” refers to the pre-miRNA, while a capitalized “miR-” refers to the mature form. miRNAs with nearly identical sequences bar one or two nucleotides are annotated with an additional lower case letter. For example, miR-123a would be closely related to miR-123b. miRNAs that are 100% identical but are encoded at different places in the genome are indicated with additional dash-number suffix: miR-123-1 and miR-123-2 are identical but are produced from different pre-miRNAs. Species of origin is designated with a three-letter prefix, e.g., hsa-miR-123 would be from human (Homo sapiens) and oar-miR-123 would be a sheep (Ovis aries) miRNA. Other common prefixes include ‘v’ for viral (miRNA encoded by a viral genome) and ‘d’ for Drosophila miRNA. microRNAs originating from the 3′ or 5′ end of a pre-miRNA are denoted with a -3p or -5p suffix. In the past, this distinction was also made with ‘s’ (sense) and ‘as’ (antisense). An asterisk following the name indicates that the miRNA is an anti-miRNA to the miRNA without an asterisk (e.g. miR-123* is an anti-miRNA to miR-123). When relative expression levels are known, an asterisk following the name indicates a miRNA expressed at low levels relative to the miRNA in the opposite arm of a hairpin. For example, miR-123 and miR-123* would share a pre-miRNA hairpin, but relatively more miR-123 would be found in the cell.

As used herein, it is understood that ‘miR-’ and ‘hsa-miR’ is used interchangeably; the results of the present invention are obtained from human samples and human miRNAs are examined.

miRBase is the central online repository for microRNA (miRNA) nomenclature, sequence data, annotation and target prediction, and may be accessed via http://www.mirbase.org/. The miRNA names used herein throughout can be accessed via this link, and specifics retrieved. See also Griffiths-Jones et al, “miRBase: tools for microRNA genomics”, Nucleic Acids Research, 2008, Vol. 36, Database issue D154-D158.

### Biomarker

A biomarker, or biological marker, is in general a substance used as an indicator of a biological state. It is a characteristic that is objectively measured and evaluated as an indicator of normal biological processes, pathogenic processes, or pharmacologic responses to a therapeutic intervention.

More specifically, a biomarker indicates a change in expression or state of a protein or miRNA that correlates with the risk or progression of a disease, or with the susceptibility of the disease to a given treatment.

A biomarker, such as a miRNA biomarker, may be correlated to a certain condition based on differences in miRNA expression levels between a test sample and a control or reference sample. If a certain miRNA biomarker is found to be deregulated in a sample as compared to a reference level, the sample has a certain probability of being associated with a certain condition.

A biomarker, such as a miRNA biomarker, may also be correlated to a certain condition based on expression levels of the biomarker at the onset of disease or during progression of disease.

According to the present invention, the miRNA biomarkers identified herein may be used to correlate the expression level of said miRNA(s) obtained from a sample from a patient with pancreatic cancer with the prognosis of said patient.

It follows that the expression of one or more miRNA biomarkers may be deregulated in a condition (e.g. cancer with poor prognosis) as compared to another condition (e.g. cancer with a better prognosis).

Prognostic miRNA Biomarkers of the Present Invention

The present invention is in one aspect directed to the provision of miRNA biomarkers that may be used to predict the prognosis of a patient with pancreatic cancer with respect to overall survival and/or 2-year follow up survival, and comprises or consists of one or more of miR-675, miR-212, miR-148a*, miR-187, let-7g*, miR-205, miR-944, miR-431, miR-194*, miR-769-5p, miR-450b-5p, miR-222, miR-146a, miR-23a*, miR-143*, miR-216a, miR-891a, miR-409-5p, miR-449b and miR-330-5p.

The present invention is in another aspect directed to the provision of miRNA biomarkers that may be used to predict the prognosis of a patient with PDAC with respect to overall survival, and comprises or consists of one or more of miR-675, miR222* and miR-29a*.

The present invention is in yet another aspect directed to the provision of miRNA biomarkers that may be used to predict the prognosis of a patient with A-AC with respect to overall survival, and comprises or consists of one or more of miR-148a and miR-625.

It is contemplated that the expression level of at least one of said miRNAs in one embodiment is measured in a sample from an individual, and said miRNA expression level is then associated with a prognosis.

Said prognosis may be defined as the predicted overall survival (OS) and/or survival at 2 years follow-up.

Said prognosis may be a graduation between ‘poor’ and ‘good’, it may be expressed in months or years expected survival, or it may be defined as a probability of surviving a certain time period expressed in months or years.

In one embodiment, the prognosis as defined herein is expressed as a probability of surviving a certain time period expressed in months or years. Said time period may be defined as 3-months survival probability, 6-months survival probability, 9-months survival probability, 12-months survival/1-year survival probability, 2-years survival probability, 3-years survival probability, 4-years survival probability, 5-years survival probability, 6-years survival probability, 7-years survival probability, 8-years survival probability, 9-years survival probability or 10-years survival probability. Said probability of survival after a certain time period may be in the range of 0.01 to 0.1, such as 0.1 to 0.2, for example 0.2 to 0.3, such as 0.3 to 0.4, for example 0.4 to 0.5, such as 0.5 to 0.6, for example 0.6 to 0.7, such as 0.7 to 0.8, for example 0.8 to 0.85, such as 0.85 to 0.9, for example 0.9 to 0.91, such as 0.91 to 0.92, for example 0.92 to 0.93, such as 0.93 to 0.94, for example 0.94 to 0.95, such as 0.95 to 0.96, for example 0.96 to 0.97, such as 0.97 to 0.98, for example 0.98 to 0.99, such as 0.99 to 1.0.

Said time period may be calculated starting from time of diagnosis, time of surgery or time of analysis/evaluation.

The 3-months survival probability may in one embodiment be between 0.9 and 1.0. The 1-year survival probability may in one embodiment be between 0.2 and 0.9. The 10-year survival probability may in one embodiment be between 0.01 and 0.6.

It follows that a probability is expressed in a value of between 0-100, where 100 is a high probability of survival the indicated time period (good prognosis), and 0 is a low probability (poor prognosis).

The miRNA biomarkers as disclosed herein may in one embodiment be used (or measured; correlated) alone.

In one embodiment, said biomarkers are used in combination (‘simple combination’), comprising at least two miRNA biomarkers. It follows that the expression level of two or more of the miRNAs according to the present invention is measured and correlated to the expected survival or prognosis.

In one embodiment, said miRNA biomarkers are used to assess the prognosis of an individual with pancreas cancer (collectively, or distinguished between PDAC and A-AC), and comprises two or more miRNAs selected from the group consisting of miR-675, miR-212, miR-148a*, miR-148a, miR-187, let-7g*, miR-205, miR-944, miR-431, miR-194*, miR-769-5p, miR-450b-5p, miR-222, miR-222*, miR-146a, miR-23a*, miR-143*, miR-216a, miR-891a, miR-409-5p, miR-449b, miR-330-5p, miR-29a* and miR-625.

It follows, that the combination of miRNA biomarkers as disclosed herein may in one embodiment consist of or comprise 2 miRNAs, such as 3 miRNAs, for example 4 miRNAs, such as 5 miRNAs, for example 6 miRNAs, such as 7 miRNAs, for example 8 miRNAs, such as 9 miRNAs, for example 10 miRNAs, such as 11 miRNAs, for example 12 miRNAs, such as 13 miRNAs, for example 14 miRNAs, such as 15 miRNAs, for example 16 miRNAs, such as 17 miRNAs, for example 18 miRNAs, such as 19 miRNAs, for example 20 miRNAs, as selected from the miRNA biomarkers disclosed herein.

In one embodiment, said miRNA biomarkers are used in combination to assess the prognosis of an individual with pancreas cancer, and comprises or consists of two or more miRNAs selected from the group consisting of
a) miR-675, miR-212, miR-148a*, miR-187, let-7g*, miR-205, miR-944, miR-431, miR-194*, miR-769-5p, miR-450b-5p, miR-222, miR-146a, miR-23a*, miR-143*, miR-216a, miR-891a, miR-409-5p, miR-449b and miR-330-5p; or
b) miR-675, miR-212, miR-148a*, miR-187 and let-7g*; or
c) miR-675, miR-212, miR-148a*, miR-187, miR-205, miR-944, miR-431, miR-194* and miR-769-5p; or
d) miR-675, miR-212, miR-148a* and miR-187; or
e) miR-675, miR-148a*, miR-450b-5p, miR-222 and miR-146a; or
f) miR-675, miR-148a*, miR-187, miR-205, miR-431, miR-194*, miR-23a*, miR-143*, miR-216a, miR-891a, miR-409-5p, miR-450b-5p, miR-449b and miR-330-5p;
g) miR-675, miR-148a* and miR-450b-5p; or
h) miR-675, miR-187, miR-205, miR-431, miR-194* and 148a*; or
i) miR-675 and miR-148a*

In another embodiment, said miRNA biomarkers are used in combination to assess the prognosis of an individual with PDAC, and comprises or consists of two or more miRNAs selected from the group consisting of miR-675, miR-222* and miR-29a*.

In another embodiment, said miRNA biomarkers are used in combination to assess the prognosis of an individual with A-AC, and comprises or consists of two miRNAs selected from the group consisting of miR-148a and miR-625*.

In one embodiment, said miRNAs alone or in combination is associated with overall survival and/or 2 year follow up survival of PC patients.

In one particular embodiment, the expression level of 1, 2, 3, 4 or 5 miRNAs selected from the group consisting of miR-675, miR-212, miR-148a*, miR-187 and let-7g* is used to evaluate the overall survival of a pancreas cancer patient.

In one particular embodiment, the expression level of 1, 2, 3, 4, 5, 6, 7, 8 or 9 miRNAs selected from the group consisting of miR-675, miR-212, miR-148a*, miR-187, miR-205, miR-944, miR-431, miR-194* and miR-769-5p is used to evaluate the overall survival of a pancreas cancer patient.

In one particular embodiment, the expression level of 1, 2, 3, 4 or 5 miRNAs selected from the group consisting of miR-675, miR-148a*, miR-450b-5p, miR-222 and miR-146a is used to evaluate the 2-year follow-up survival of a pancreas cancer patient.

In one particular embodiment, the expression level of 1, 2, 3, 4, 5, 6, 7, 8, 9, 10, 11, 12, 13 or 14 miRNAs selected from the group consisting of miR-675, miR-148a*, miR-187, miR-205, miR-431, miR-194*, miR-23a*, miR-143*, miR-216a, miR-891a, miR-409-5p, miR-450b-5p, miR-449b and miR-330-5p is used to evaluate the 2-year follow-up survival of a pancreas cancer patient.

In a particular embodiment, high expression levels of one or more of miR-675 and miR-212, and low expression levels of one or more of miR-148a*, miR-187 and let-7g*, are predictors of (or associated with) short overall survival.

In a particular embodiment, high expression levels of one or more of miR-675, miR-450b-5p and miR-222, and low expression levels of one or more of miR-148a* and miR-146a, are predictors of (or associated with) poor 2-years survival.

In a particular embodiment, high expression levels of one or more of miR-675, miR-miR-222* and low expression levels of miR-29a* are predictors of (or associated with) poor overall survival for the subgroup of PDAC pancreatic cancer patients.

In a particular embodiment, low expression levels of one or more of miR-148a and miR-625 are predictors of (or associated with) poor overall survival for the subgroup of A-AC pancreatic cancer patients.

The combination of miRNA biomarkers as disclosed may in another embodiment consist of or comprise between 2 to 3 miRNAs of the present invention, such as between 3 to 4 miRNAs, for example between 4 to 5 miRNAs, such as between 5 to 6 miRNAs, for example between 6 to 7 miRNAs, such as between 7 to 8 miRNAs, for example between 8 to 9 miRNAs, such as between 9 to 10 miRNAs, for example between 10 to 11 miRNAs, such as between 11 to 12 miRNAs, for example between 12 to 13 miRNAs, such as between 13 to 14 miRNAs, for example between 14 to 15 miRNAs, such as between 15 to 16 miRNAs, for example between 16 to 17 miRNAs, such as between 17 to 18 miRNAs, for example between 18 to 19 miRNAs, such as between 19 to 20 miRNAs of the present invention.

In a particular embodiment, the miRNA biomarker according to the present invention is not selected from the group consisting of miR-187, miR-205 and miR-222.

In a particular embodiment, miR-187, miR-205 and miR-222 are used according to the present invention only in combination with one or more additional miRNAs selected from those identified herein.

In one embodiment, the combination of miRNA biomarkers to predict overall survival and/or 2-years survival comprises at least miR-675. In one embodiment, the combination of miRNA biomarkers to predict overall survival and/or 2-years survival comprises at least let-7g*. In one embodiment, the combination of miRNA biomarkers to predict overall survival and/or 2-years survival comprises at least miR-148a*.

### Classifier

Classifiers are relationships between sets of input variables, usually known as features, and discrete output variables, known as classes. Classes are often centered on the key questions of who, what, where and when. A classifier can intuitively be thought of as offering an opinion about whether, for instance, an individual associated with a given feature set is a member of a given class.

In other words, a classifier is a predictive model that attempts to describe one column (the label) in terms of others (the attributes). A classifier is constructed from data where the label is known, and may be later applied to predict label values for new data where the label is unknown. Internally, a classifier is an algorithm or mathematical formula that predicts one discrete value for each input row. For example, a classifier built from a dataset of iris flowers could predict the type of a presented iris given the length and width of its petals and stamen. Classifiers may also produce probability estimates for each value of the label. For example, a classifier built from a dataset of cars could predict the probability that a specific car was built in the United States.

miRNA Classifier of the Present Invention

The miRNA classifiers according to the present invention are the relationships between sets of input variables, i.e. the miRNA expression in a sample of an individual, and discrete output variables, i.e. distinction between two conditions e.g. poor or good survival. Thus, the classifier assigns a given sample to a given class with a given probability.

Distinction, differentiation or characterisation of a sample is used herein as being capable of predicting with a high sensitivity and specificity if a given sample of unknown prognosis belongs to one of two classes (two-way classifier).

In one aspect, the miRNA classifier is a two-way classifier capable of predicting with an adequate sensitivity and specificity if a given sample of unknown prognosis has a certain probability of being associated with a specific predicted survival, wherein said miRNA classifier comprises or consists of one or more miRNAs selected from the group consisting of miR-675, miR-212, miR-148a*, miR-148a, miR-187, let-7g*, miR-205, miR-944, miR-431, miR-194*, miR-769-5p, miR-450b-5p, miR-222, miR-222*, miR-146a, miR-23a*, miR-143*, miR-216a, miR-891a, miR-409-5p, miR-449b, miR-330-5p, miR-29a* and miR-625.

Said specific predicted survival may be expressed as the probability for surviing at 3-months, 6-months, 9-months, 12-months/1-year, 2-years, 3-years, 4-years, 5-years, 6-years, 7-years, 8-years, 9-years or 10-years; calculated from time of diagnosis, time of surgery or time of analysis/evaluation.

In one embodiment, said miRNA classifier comprises or consists of the group of miR-675, miR-212, miR-148a*, miR-187, miR-205, miR-944, miR-431, miR-194* and miR-769-5p, and is telling of the overall survival of the patient.

In one embodiment, said miRNA classifier comprises or consists of the group of miR-675, miR-148a*, miR-187, miR-205, miR-431, miR-194*, miR-23a*, miR-143*, miR-216a, miR-891a, miR-409-5p, miR-450b-5p, miR-449b and miR-330-5p, and is telling of the 2-years survival of the patient.

The output of the two-way miRNA classifier is given as a probability of belonging to either class of between 0-1 (prediction probability). If the value for a sample is 0.5, no prediction is made. A number or value of between 0.51 to 1.0 for a given sample means that the sample is predicted to belong to the class in question, e.g. NP; and the corresponding value of 0.0 to 0.49 for the second class in question, e.g. PC, means that the sample is predicted not to belong to the class in question.

In one embodiment, the prediction probabilities for a sample to belong to a certain class is a number falling in the range of from 0 to 1, such as from 0.0 to 0.1, for example 0.1 to 0.2, such as 0.2 to 0.3, for example 0.3 to 0.4, such as 0.4 to 0.49, for example 0.5, such as 0.51 to 0.6, for example 0.6 to 0.7, such as 0.7 to 0.8, for example 0.8 to 0.9, such as 0.9 to 1.0.

The classifier according to the present invention may in one embodiment comprise or consist of 2 miRNAs, such as 3 miRNAs, for example 4 miRNAs, such as 5 miRNAs, for example 6 miRNAs, such as 7 miRNAs, for example 8 miRNAs, such as 9 miRNAs, for example 10 miRNAs, such as 11 miRNAs, for example 12 miRNAs, such as 13 miRNAs, for example 14 miRNAs, such as 15 miRNAs, for example 16 miRNAs, such as 17 miRNAs, for example 18 miRNAs, such as 19 miRNAs, for example 20 miRNAs selected from the group consisting of miR-675, miR-212, miR-148a*, miR-148a, miR-187, let-7g*, miR-205, miR-944, miR-431, miR-194*, miR-769-5p, miR-450b-5p, miR-222, miR-222*, miR-146a, miR-23a*, miR-143*, miR-216a, miR-891a, miR-409-5p, miR-449b, miR-330-5p, miR-29a* and miR-625.

Methods for Evaluating the Prognosis of an Individual with Pancreas Cancer

The present invention in one aspect relates to a method for predicting the prognosis for a patient with pancreatic cancer, said method comprising measuring the expression level of at least one miRNA in a sample obtained from said individual, wherein the at least one miRNA is selected from the group consisting of
a) miR-675, miR-212, miR-148a*, miR-187, let-7g*, miR-205, miR-944, miR-431, miR-194*, miR-769-5p, miR-450b-5p, miR-222, miR-146a, miR-23a*, miR-143*, miR-216a, miR-891a, miR-409-5p, miR-449b and miR-330-5p; or
b) miR-675, miR-212, miR-148a*, miR-187 and let-7g*; or
c) miR-675, miR-212, miR-148a*, miR-187, miR-205, miR-944, miR-431, miR-194* and miR-769-5p; or
d) miR-675, miR-212, miR-148a* and miR-187; or
e) miR-675, miR-148a*, miR-450b-5p, miR-222 and miR-146a; or
f) miR-675, miR-148a*, miR-187, miR-205, miR-431, miR-194*, miR-23a*, miR-143*, miR-216a, miR-891a, miR-409-5p, miR-450b-5p, miR-449b and miR-330-5p;
g) miR-675, miR-148a* and miR-450b-5p; or
h) miR-675, miR-187, miR-205, miR-431, miR-194* and 148a*; or
i) miR-675 and miR-148a*, or
j) miR-675, miR-212, miR-148a* and let-7g*.

The present invention in another aspect relates to a method for predicting the prognosis for a patient with PDAC, said method comprising measuring the expression level of at least one miRNA in a sample obtained from said individual, wherein the at least one miRNA is selected from the group consisting of miR-675, miR222* and miR-29a*.

The present invention in another aspect relates to a method for predicting the prognosis for a patient with A-AC, said method comprising measuring the expression level of at least one miRNA in a sample obtained from said individual, wherein the at least one miRNA is selected from the group consisting of miR-148a* and miR-625.

In one embodiment, said method is a method for estimating the probability for a patient with pancreatic cancer of surviving for a certain time period, wherein the miRNA expression level of at least one of said miRNAs is indicative of said individual with pancreatic carcinoma surviving for a certain time period.

The present invention in one aspect thus relates to a method for estimating the probability for a patient with pancreatic cancer of surviving for a certain time period, said method comprising measuring the expression level of at least one miRNA in a sample obtained from said individual, wherein the at least one miRNA is selected from the group consisting of
a) miR-675, miR-212, miR-148a*, miR-187, let-7g*, miR-205, miR-944, miR-431, miR-194*, miR-769-5p, miR-450b-5p, miR-222, miR-146a, miR-23a*, miR-143*, miR-216a, miR-891a, miR-409-5p, miR-449b and miR-330-5p; or
b) miR-675, miR-212, miR-148a*, miR-187 and let-7g*; or
c) miR-675, miR-212, miR-148a*, miR-187, miR-205, miR-944, miR-431, miR-194* and miR-769-5p; or
d) miR-675, miR-212, miR-148a* and miR-187; or
e) miR-675, miR-148a*, miR-450b-5p, miR-222 and miR-146a; or
f) miR-675, miR-148a*, miR-187, miR-205, miR-431, miR-194*, miR-23a*, miR-143*, miR-216a, miR-891a, miR-409-5p, miR-450b-5p, miR-449b and miR-330-5p;
g) miR-675, miR-148a* and miR-450b-5p; or
h) miR-675, miR-187, miR-205, miR-431, miR-194* and 148a*; or
i) miR-675 and miR-148a*; or
j) miR-675, miR-212, miR-148a* and let-7g*; or
k) miR-675, miR-222* and miR-29a*; or
l) miR-148a and miR-625
   wherein the miRNA expression level of at least one of said miRNAs is indicative of said individual with pancreatic carcinoma surviving for a certain time period.

In one embodiment, said method further comprises the step of determining the probability for said individual with pancreatic carcinoma of surviving for the indicated time period. Said probability of surviving for a certain time period may be in the range of 0.01 to 0.1, such as 0.1 to 0.2, for example 0.2 to 0.3, such as 0.3 to 0.4, for example 0.4 to 0.5, such as 0.5 to 0.6, for example 0.6 to 0.7, such as 0.7 to 0.8, for example 0.8 to 0.85, such as 0.85 to 0.9, for example 0.9 to 0.91, such as 0.91 to 0.92, for example 0.92 to 0.93, such as 0.93 to 0.94, for example 0.94 to 0.95, such as 0.95 to 0.96, for example 0.96 to 0.97, such as 0.97 to 0.98, for example 0.98 to 0.99, such as 0.99 to 1.0.

In one embodiment, said time period may be expressed as 3-months survival probability, 6-months survival probability, 9-months survival probability, 12-months survival/1-year survival probability, 2-years survival probability, 3-years survival probability, 4-years survival probability, 5-years survival probability, 6-years survival probability, 7-years survival probability, 8-years survival probability, 9-years survival probability or 10-years survival probability.

Said time period may be calculated starting from time of diagnosis, time of surgery or time of analysis/evaluation.

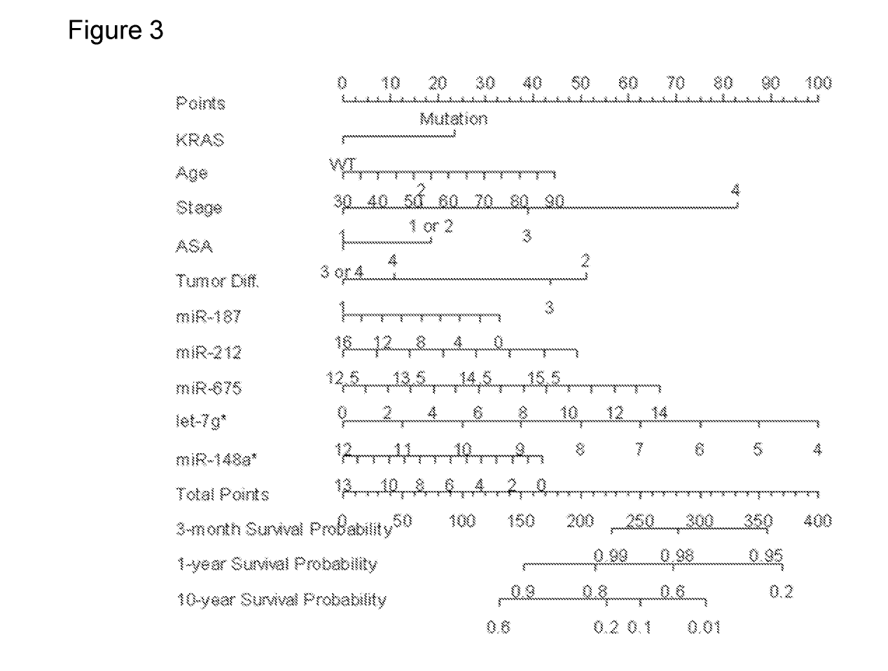

In one particular embodiment, the step of determining the probability for said individual with pancreatic carcinoma of surviving for an indicated time period is performed by employing a nomogram, such as the nomogram depicted in FIG. 3 herein.

A nomogram, nomograph, or abac is a graphical calculating device, a two-dimensional diagram designed to allow the approximate graphical computation of a function. Defining alternatively, a nomogram is a (two-dimensionally) plotted function with n parameters, from which, knowing n−1 parameters, the unknown one can be read, or fixing some parameters, the relationship between the unfixed ones can be studied.

In one embodiment, said method further comprises the step of correlating the miRNA expression level of at least one of said miRNAs to a predetermined reference level.

In one embodiment, said method further comprises the step of obtaining a sample from an individual with pancreas cancer, by any means as disclosed herein elsewhere.

In one embodiment, said method further comprises the step of extracting RNA from a sample collected from an individual with pancreas cancer, by any means as disclosed herein elsewhere.

Said sample is in one particular embodiment a tissue sample from the pancreas of said individual. In another embodiment, said sample is a blood sample from said individual.

In one embodiment, said miRNA expression level is altered as compared to the expression level in a reference sample. Said reference sample may in one embodiment be a sample from a patient with a known estimated prognosis.

In one embodiment, the prognosis as defined herein is expressed as a probability of surviving a certain time period expressed in months or years. Said time period may be defined as 3-months survival probability, 6-months survival probability, 9-months survival probability, 12-months survival/1-year survival probability, 2-years survival probability, 3-years survival probability, 4-years survival probability, 5-years survival probability, 6-years survival probability, 7-years survival probability, 8-years survival probability, 9-years survival probability or 10-years survival probability.

In one embodiment, said pancreatic carcinoma is pancreatic adenocarcinoma. In another embodiment, said pancreatic carcinoma is ampullary adenocarcinoma. In a further embodiment, said pancreatic carcinoma comprises both pancreatic adenocarcinoma and ampullary adenocarcinoma.

In one embodiment, the at least one miRNA according to the above method comprises or consists of miR-675, miR-212, miR-148a*, miR-187, let-7g*, miR-205, miR-944, miR-431, miR-194*, miR-769-5p, miR-450b-5p, miR-222, miR-146a, miR-23a*, miR-143*, miR-216a, miR-891a, miR-409-5p, miR-449b and miR-330-5p.

In one embodiment, the at least one miRNA according to the above method comprises or consists of miR-675, miR-212, miR-148a*, miR-187 and let-7g*.

In one embodiment, the at least one miRNA according to the above method comprises or consists of miR-675, miR-212, miR-148a*, miR-187, miR-205, miR-944, miR-431, miR-194* and miR-769-5p.

In one embodiment, the at least one miRNA according to the above method comprises or consists of miR-675, miR-212, miR-148a* and miR-187.

In one embodiment, the at least one miRNA according to the above method comprises or consists of miR-675, miR-148a*, miR-450b-5p, miR-222 and miR-146a.

In one embodiment, the at least one miRNA according to the above method comprises or consists of miR-675, miR-148a*, miR-187, miR-205, miR-431, miR-194*, miR-23a*, miR-143*, miR-216a, miR-891a, miR-409-5p, miR-450b-5p, miR-449b and miR-330-5p.

In one embodiment, the at least one miRNA according to the above method comprises or consists of miR-675, miR-148a* and miR-450b-5p.

In one embodiment, the at least one miRNA according to the above method comprises or consists of miR-187, miR-205, miR-431, miR-194* and 148a*.

In one embodiment, the at least one miRNA according to the above method comprises or consists of miR-675 and miR-148a*.

In one embodiment, the at least one miRNA according to the above method comprises or consists of miR-675, miR-212, miR-148a* and let-7g*.

In one embodiment, the at least one miRNA according to the above method comprises or consists of miR-675, miR222* and miR-29a*.

In one embodiment, the at least one miRNA according to the above method comprises or consists of miR-148a* and miR-625.

In one embodiment, the at least one miRNA according to the above method consists of the group consisting of miR-675, miR-212, miR-148a* and miR-187; or the group consisting of miR-675, miR-212, miR-148a* and let-7g*; or the group consisting of miR-675, miR-212, miR-187 and let-7g*; or the group consisting of miR-675, miR-148a*, miR-187 and let-7g*; or the group consisting of miR-212, miR-148a*, miR-187 and let-7g*; or the group consisting of miR-675, miR-212 and miR-148a*; or the group consisting of miR-675, miR-212 and miR-187; or the group consisting of miR-675, miR-212 and let-7g*; or the group consisting of miR-675, miR-148a* and miR-187; or the group consisting of miR-675, miR-148a* and let-7g*; or the group consisting of miR-675, miR-187 and let-7g*; or the group consisting of miR-212, miR-148a* and miR-187; or the group consisting of miR-212, miR-148a* and let-7g*; or the group consisting of miR-212, miR-187 and let-7g*; or the group consisting of miR-148a*, miR-187 and let-7g*; or the group consisting of miR-675 and miR-212; or the group consisting of miR-675 and miR-148a*; or the group consisting of miR-675 and miR-187; or the group consisting of miR-675 and let-7g*; or the group consisting of miR-212 and miR-148a*; or the group consisting of miR-212 and miR-187; or the group consisting of miR-212 and miR-187; or the group consisting of miR-148a* and let-7g*; or the group consisting of miR-148a* and miR-187; or the group consisting of miR-187 and let-7g*.

It follows, that any of the above-mentioned methods may further comprise the step of obtaining prediction probabilities of between 0-1.

In one embodiment, said method for predicting the prognosis for a patient with pancreatic cancer comprises measuring the expression level of at least 2 miRNAs; for example 2 miRNAs, such as 3 miRNAs, for example 4 miRNAs, such as 5 miRNAs, for example 6 miRNAs, such as 7 miRNAs, for example 8 miRNAs, such as 9 miRNAs, for example 10 miRNAs, such as 11 miRNAs, for example 12 miRNAs, such as 13 miRNAs, for example 14 miRNAs, such as 15 miRNAs, for example 16 miRNAs, such as 17 miRNAs, for example 18 miRNAs, such as 19 miRNAs, for example 20 miRNAs, as selected from the prognostic miRNAs as disclosed herein.

In one embodiment, said method of predicting the prognosis for a patient with pancreatic cancer further comprises measuring the expression level of one or more additional miRNAs, besides or not comprising the group of miR-675, miR-212, miR-148a*, miR-187, let-7g*, miR-205, miR-944, miR-431, miR-194*, miR-769-5p, miR-450b-5p, miR-222, miR-146a, miR-23a*, miR-143*, miR-216a, miR-891a, miR-409-5p, miR-449b and miR-330-5p.

Thus, said method of predicting a prognosis further comprises measuring the expression level of one or more additional miRNAs which have previously been identified in the literature as having prognostic value.

In one embodiment, said method of predicting a prognosis further comprises measuring the expression level of one or more additional miRNAs selected from the group of miR-21, miR-155, miR-187, miR-222, miR-203, miR-452, miR-105, miR-127, miR-518a-2, miR-30a-3p.

In a further embodiment, any of the above-mentioned methods may be is used in combination with at least one additional prognostic method, which may improve the sensitivity and/or specificity and/or accuracy of the combined prognostic outcome.

The invention in a further aspect relates to a method for expression profiling of a sample obtained from a pancreas cancer patient, said method comprising measuring at least one miRNA selected from the group of
a) miR-675, miR-212, miR-148a*, miR-187, let-7g*, miR-205, miR-944, miR-431, miR-194*, miR-769-5p, miR-450b-5p, miR-222, miR-146a, miR-23a*, miR-143*, miR-216a, miR-891a, miR-409-5p, miR-449b and miR-330-5p; or
b) miR-675, miR-212, miR-148a*, miR-187 and let-7g*; or
c) miR-675, miR-212, miR-148a*, miR-187, miR-205, miR-944, miR-431, miR-194* and miR-769-5p; or
d) miR-675, miR-212, miR-148a* and miR-187; or
e) miR-675, miR-148a*, miR-450b-5p, miR-222 and miR-146a; or
f) miR-675, miR-148a*, miR-187, miR-205, miR-431, miR-194*, miR-23a*, miR-143*, miR-216a, miR-891a, miR-409-5p, miR-450b-5p, miR-449b and miR-330-5p;
g) miR-675, miR-148a* and miR-450b-5p; or
h) miR-675, miR-187, miR-205, miR-431, miR-194* and 148a*; or
i) miR-675 and miR-148a*; or
j) miR-675, miR-212, miR-148a* and let-7g*; or
k) miR-675, miR-222* and miR-29a*; or
l) miR-148a and miR-625
   and correlating said expression profile to the expected survival of a patient with pancreatic cancer.

### Sample Type

The sample according to the present invention is extracted from an individual and used for miRNA expression profiling for the subsequent assessment of the prognosis of an individual having been diagnosed with pancreas cancer.

The sample may be collected from an individual or a cell culture, preferably an individual. The individual may be any animal, such as a mammal, including human beings. In a preferred embodiment, the individual is a human being.

In a particular embodiment, the sample is taken from the pancreas of a human being. In such an instance, the sample may be denoted a tissue sample. Said pancreas sample preferably comprises pancreatic cells, preferably pancreatic cancer cells.

The tissue sample may further comprise cells of the desmoplastic stroma surrounding the pancreatic tumour, e.g. fibroblasts, pancreatic stellate cells, inflammatory cells (e.g. macrophages and neutrofils) and endothelial cells.

In another particular embodiment, the sample is a blood sample drawn from a human being.

### Sample Collection

In one embodiment, the sample is collected from the pancreas of an individual by any available means.

In one embodiment, the sample is collected from the pancreas by fine-needle aspiration (FNA) using a needle with a maximum diameter of 1 mm; by core needle aspiration using a needle with a maximum diameter of above 1 mm (also called coarse needle aspiration or biopsy, large needle aspiration or large core aspiration); by biopsy; by cutting biopsy; by open biopsy; a surgical sample; or by any other means known to the person skilled in the art. In another embodiment, the sample is collected from an in vitro cell culture.

In a particular embodiment, the sample is obtained by surgery, such as by surgery with radical intentions.

In a particular embodiment, the sample is a fine-needle aspirate from an individual. The fine-needle aspiration may be performed using a needle with a diameter of between 0.2 to 1.0 mm, such as 0.2 to 0.3 mm, for example 0.3 to 0.4 mm, such as 0.4 to 0.5 mm, for example 0.5 to 0.6 mm, such as 0.6 to 0.7 mm, for example 0.7 to 0.8 mm, such as 0.8 to 0.9 mm, for example 0.9 to 1.0 mm in diameter.

Said fine-needle aspiration may in one embodiment be a single fine-needle aspiration, or may in another embodiment comprise multiple fine-needle aspirations.

The diameter of the needle is indicated by the needle gauge. Various needle lengths are available for any given gauge. Needles in common medical use range from 7 gauge (the largest) to 33 (the smallest) on the Stubs scale. Although reusable needles remain useful for some scientific applications, disposable needles are far more common in medicine. Disposable needles are embedded in a plastic or aluminium hub that attaches to the syringe barrel by means of a press-fit (Luer) or twist-on (Luer-lock) fitting.

The fine-needle aspiration is in one embodiment performed using a needle gauge of between 20 to 33, such as needle gauge 20, for example needle gauge 21, such as needle gauge 22, for example needle gauge 23, such as needle gauge 24, for example needle gauge 25, such as needle gauge 26, for example needle gauge 27, such as needle gauge 28, for example needle gauge 29, such as needle gauge 30, for example needle gauge 31, such as needle gauge 32, for example needle gauge 33.

The fine-needle aspiration may in one embodiment be assisted, such as ultra-sound (US) guided fine-needle aspiration, x-ray guided fine-needle aspiration, endoscopic ultra-sound (EUS) guided fine-needle aspiration, Endobronchial ultrasound-guided fine-needle aspiration (EBUS), ultrasonographically guided fine-needle aspiration, stereotactically guided fine-needle aspiration, computed tomography (CT)—guided percutaneous fine-needle aspiration and palpation guided fine-needle aspiration.

The skin above the area to be biopsied may in one embodiment be swiped with an antiseptic solution and/or may be draped with sterile surgical towels. The skin, underlying fat, and muscle may in one embodiment be numbed with a local anesthetic. After the needle is placed into the mass, cells may be withdrawn by aspiration with a syringe.

In another embodiment, the sample is a blood sample extracted or drawn from an individual by any conventional method known to the skilled person. The blood may be drawn from a vein or an artery of an individual.

The sample extracted from an individual by any means as disclosed above may be transferred to a tube or container prior to analysis. The container may be empty, or may comprise a collection media of sorts.

The sample extracted from an individual by any means as disclosed above may be analysed essentially immediately, or it may be stored prior to analysis for a variable period of time and at various temperature ranges.

In one embodiment, the sample is stored at a temperature of between −200° C. to 37° C., such as between −200 to −100° C., for example −100 to −50° C., such as −50 to −25° C., for example—25 to −10° C., such as −10 to 0° C., for example 0 to 10° C., such as 10 to 20° C., for example 20 to 30° C., such as 30 to 37° C. prior to analysis.

In one embodiment, the sample is stored at −20° C. and/or −80° C.

In another embodiment, the sample is stored for between 15 minutes and 100 years prior to analysis, such as between 15 minutes and 1 hour, for example 1 to 2 hours, such as 2 to 5 hours, for example 5 to 10 hours, such as 10 to 24 hours, for example 24 hours to 48 hours, such as 48 to 72 hours, for example 72 to 96 hours, such as 4 to 7 days, such as 1 week to 2 weeks, such as 2 to 4 weeks, such as 4 weeks to 1 month, such as 1 month to 2 months, for example 2 to 3 months, such as 3 to 4 months, for example 4 to 5 months, such as 5 to 6 months, for example 6 to 7 months, such as 7 to 8 months, for example 8 to 9 months, such as 9 to 10 months, for example 10 to 11 months, such as 11 to 12 months, for example 1 year to 2 years, such as 2 to 3 years, for example 3 to 4 years, such as 4 to 5 years, for example 5 to 6 years, such as 6 to 7 years, for example 7 to 8 years, such as 8 to 9 years, for example 9 to 10 years, such as 10 to 20 years, for example 20 to 30 years, such as 30 to 40 years, for example 40 to 50 years, such as 50 to 75 years, for example 75 to 100 years prior to analysis.

In one embodiment, the sample is stored for a few days.

### Collection Media for Sample

A collection media according to the present invention is any media suitable for preserving and/or collecting a sample for immediate or later analysis.

In one embodiment, said collection media is a solution suitable for sample preservation and/or later retrieval of RNA (such as miRNA) from said sample.

In one embodiment, the collection media is an RNA preservation solution or reagent suitable for containing samples without the immediate need for cooling or freezing the sample, while maintaining RNA integrity prior to extraction of RNA (such as miRNA) from the sample. An RNA preservation solution or reagent may also be known as RNA stabilization solution or reagent or RNA recovery media, and may be used interchangeably herein. The RNA preservation solution may penetrate the harvested cells of the collected sample to retard RNA degradation to a rate dependent on the storage temperature.

The RNA preservation solution may be any commercially available solutions or it may be a solution prepared according to available protocols.

The commercially available RNA preservation solutions may for example be selected from RNAlater® (Ambion and Qiagen), PreservCyt medium (Cytyc Corp), PrepProtect™ Stabilisation Buffer (Miltenyi Biotec), Allprotect Tissue Reagent (Qiagen) and RNAprotect Cell Reagent (Qiagen). Protocols for preparing a RNA stabilizing solution may be retrieved from the internet (e.g. L. A. Clarke and M. D. Amaral: ‘Protocol for RNase-retarding solution for cell samples’, provided through The European Workin Group on CFTR Expression), or may be produced and/or optimized according to techniques known to the skilled person.

In another embodiment, the collection media will penetrate and lyse the cells of the sample immediately, including reagents and methods for isolating RNA (such as miRNA) from a sample that may or may not include the use of a spin column. Other collection media according to the present invention comprises any media such as water, sterile water, denatured water, saline solutions, buffers, PBS, TBS, Allprotect Tissue Reagent (Qiagen), cell culture media such as RPMI-1640, DMEM (Dulbecco's Modified Eagle Medium), MEM (Minimal Essential Medium), IMDM (Iscove's Modified Dulbecco's Medium), BGjB (Fitton-Jackson modification), BME (Basal Medium Eagle), Brinster's BMOC-3 Medium, CMRL Medium, CO₂-Independent Medium, F-10 and F-12 Nutrient Mixture, GMEM (Glasgow Minimum Essential Medium), IMEM (Improved Minimum Essential Medium), Leibovitz's L-15 Medium, McCoy's 5A Medium, MCDB 131 Medium, Medium 199, Opti-MEM, Waymouth's MB 752/1, Williams' Media E, Tyrode's solution, Belyakov's solution, Hanks' solution and other cell culture media known to the skilled person, tissue preservation media such as HypoThermosol®, CryoStor™ and Steinhardt's medium and other tissue preservation media known to the skilled person.

In another embodiment, said collection media is means for fixation (preservation) of said tissue sample; a tissue fixative, such as formalin (formaldehyde) or the like.

Types of tissue fixation includes heat fixation, chemical fixation (Crosslinking fixatives—Aldehydes; Precipitating fixatives—Alcohols; Oxidising agents; Mercurials; Picrates; HOPE (Hepes-glutamic acid buffer-mediated organic solvent protection effect) Fixative), and Frozen Sections.

In one embodiment, the fixation time may be between 1 to 7 calendar days; such as 1 day, 2 days, 3 days, 4 days, 5 days, 6 days or 7 days.

It follows that the invention may be carried out on formalin fixed paraffin embedded tissue blocks (FFPE).

### Sample Analysis

After the sample is collected, it is subjected to analysis. In one embodiment, the sample is initially used for isolating or extracting RNA according to any conventional methods known in the art; followed by an analysis of the miRNA expression in said sample.

### Extraction of RNA

The RNA isolated from the sample may be total RNA, mRNA, microRNA, tRNA, rRNA or any type of RNA.

Conventional methods and reagents for isolating RNA from a sample comprise High Pure miRNA Isolation Kit (Roche), Trizol (Invitrogen), Guanidinium thiocyanate-phenol-chloroform extraction, PureLink™ miRNA isolation kit (Invitrogen), PureLink Micro-to-Midi Total RNA Purification System (invitrogen), RNeasy kit (Qiagen), miRNeasy kit (Qiagen), Oligotex kit (Qiagen), phenol extraction, phenol-chloroform extraction, TCA/acetone precipitation, ethanol precipitation, Column purification, Silica gel membrane purification, PureYield™ RNA Midiprep (Promega), PolyATtract System 1000 (Promega), Maxwell® 16 System (Promega), SV Total RNA Isolation (Promega), geneMAG-RNA/DNA kit (Chemicell), TRI Reagent® (Ambion), RNAqueous Kit (Ambion), ToTALLY RNA™ Kit (Ambion), Poly(A)Purist™ Kit (Ambion) and any other methods, commercially available or not, known to the skilled person.

If the sample is a FFPE, the tissue sections are initially deparaffinised, such as in xylene and ethanol.

The RNA may be further amplified, cleaned-up, concentrated, DNase treated, quantified or otherwise analysed or examined such as by agarose gel electrophoresis, absorbance spectrometry or Bioanalyser analysis (Agilent) or subjected to any other post-extraction method known to the skilled person.

Methods for extracting and analysing an RNA sample are disclosed in Molecular Cloning, A Laboratory Manual (Sambrook and Russell (ed.), 3^{rd} edition (2001), Cold Spring Harbor Laboratory Press, Cold Spring Harbor, N.Y., USA.

### RT-QPCR

The isolated RNA may be analysed by quantitative (‘real-time’) PCR (QPCR). In one embodiment, the expression level of one or more miRNAs is determined by the quantitative polymerase chain reaction (QPCR) technique.

Real-time polymerase chain reaction, also called quantitative polymerase chain reaction (Q-PCR/qPCR/RT-QPCR) or kinetic polymerase chain reaction, is a technique based on the polymerase chain reaction, which is used to amplify and simultaneously quantify a targeted DNA molecule. It enables both detection and quantification (as absolute number of copies or relative amount when normalized to DNA input or additional normalizing genes) of a specific sequence in a DNA sample.

The procedure follows the general principle of polymerase chain reaction; its key feature is that the amplified DNA is quantified as it accumulates in the reaction in real time after each amplification cycle. Two common methods of quantification are the use of fluorescent dyes that intercalate with double-stranded DNA, and modified DNA oligonucleotide probes that fluoresce when hybridized with a complementary DNA. Frequently, real-time polymerase chain reaction is combined with reverse transcription polymerase chain reaction to quantify low abundance messenger RNA (mRNA), or miRNA, enabling a researcher to quantify relative gene expression at a particular time, or in a particular cell or tissue type.

In a real time PCR assay a positive reaction is detected by accumulation of a fluorescent signal. The Ct (cycle threshold) is defined as the number of cycles required for the fluorescent signal to cross the threshold (i.e. exceeds background level). Ct levels are inversely proportional to the amount of target nucleic acid in the sample (i.e. the lower the Ct level the greater the amount of target nucleic acid in the sample). Most real time assays undergo 40 cycles of amplification.

Cts<29 are strong positive reactions indicative of abundant target nucleic acid in the sample. Cts of 30-37 are positive reactions indicative of moderate amounts of target nucleic acid. Cts of 38-40 are weak reactions indicative of minimal amounts of target nucleic acid which could represent an infection state or environmental contamination. The QPCR may be performed using chemicals and/or machines from a commercially available platform.

The QPCR may be performed using QPCR machines from any commercially available platform; such as Prism, geneAmp or StepOne Real Time PCR systems (Applied Biosystems), LightCycler (Roche), RapidCycler (Idaho Technology), MasterCycler (Eppendorf), iCycler iQ system, Chromo 4 system, CFX, MiniOpticon and Opticon systems (Bio-Rad), SmartCycler system (Cepheid), RotorGene system (Corbett Lifescience), MX3000 and MX3005 systems (Stratagene), DNA Engine Opticon system (Qiagen), Quantica qPCR systems (Techne), InSyte and Syncrom cycler system (BioGene), DT-322 (DNA Technology), Exicycler Notebook Thermal cycler, TL998 System (lanlong), Line-Gene-K systems (Bioer Technology), or any other commercially available platform.

The QPCR may be performed using chemicals from any commercially available platform, such as NCode EXPRESS qPCR or EXPRESS qPCR (Invitrogen), Taqman or SYBR green qPCR systems (Applied Biosystems), Real-Time PCR reagents (Eurogentec), iTaq mix (Bio-Rad), qPCR mixes and kits (Biosense), and any other chemicals, commercially available or not, known to the skilled person.

The QPCR reagents and detection system may be probe-based, or may be based on chelating a fluorescent chemical into double-stranded oligonucleotides.

The QPCR reaction may be performed in a tube; such as a single tube, a tube strip or a plate, or it may be performed in a microfluidic card in which the relevant probes and/or primers are already integrated.

A Microfluidic card allows high throughput, parallel analysis of mRNA or miRNA expression patterns, and allows for a quick and cost-effective investigation of biological pathways. The microfluidic card may be a piece of plastic that is riddled with micro channels and chambers filled with the appropriate probes. A sample in fluid form is injected into one end of the card, and capillary action causes the fluid sample to be distributed into the microchannels. The microfluidic card is then placed in an appropriate device for processing the card and reading the signal.

Any commercially available (predesigned or custom-made) microfluidic card may be used. Said microfluidic card may comprise a number of probes and/or primers for analysing the expression of a number of miRNAs, such as between 1-10 miRNAs, for example 10-20 miRNA, such as between 20-30 miRNAs, for example 30-40 miRNA, such as between 40-50 miRNAs, for example 50-100 miRNA, such as between 100-200 miRNAs, for example 200-300 miRNA, such as between 300-400 miRNAs, for example 400-500 miRNA, such as between 500-1000 miRNAs.

In one embodiment, the microfluidic card are TaqMan® Array Human MicroRNA A+B Cards V2.0 (Applied Biosystems).

### Microarray Analysis

The isolated RNA may be analysed by microarray analysis. In one embodiment, the expression level of one or more miRNAs is determined by the microarray technique.

A microarray is a multiplex technology that consists of an arrayed series of thousands of microscopic spots of DNA oligonucleotides or antisense miRNA probes, called features, each containing picomoles of a specific oligonucleotide sequence. This can be a short section of a gene or other DNA or RNA element that are used as probes to hybridize a DNA or RNA sample (called target) under high-stringency conditions. Probe-target hybridization is usually detected and quantified by fluorescence-based detection of fluorophore-labeled targets to determine relative abundance of nucleic acid sequences in the target. In standard microarrays, the probes are attached to a solid surface by a covalent bond to a chemical matrix (via epoxy-silane, amino-silane, lysine, polyacrylamide or others). The solid surface can be glass or a silicon chip, in which case they are commonly known as gene chip. DNA arrays are so named because they either measure DNA or use DNA as part of its detection system. The DNA probe may however be a modified DNA structure such as LNA (locked nucleic acid).

In one embodiment, the microarray analysis is used to detect microRNA, known as microRNA or miRNA expression profiling.

The microarray for detection of microRNA may be a microarray platform, wherein the probes of the microarray may be comprised of antisense miRNAs or DNA oligonucleotides. In the first case, the target is a labelled sense miRNA sequence, and in the latter case the miRNA has been reverse transcribed into cDNA and labelled.

The microarray for detection of microRNA may be a commercially available array platform, such as NCode™ miRNA Microarray Expression Profiling (Invitrogen), miRCURY LNA™ microRNA Arrays (Exiqon), microRNA Array (Agilent), μParaflo® Microfluidic Biochip Technology (LC Sciences), MicroRNA Profiling Panels (Illumina), Geniom® Biochips (Febit Inc.), microRNA Array (Oxford Gene Technology), Custom AdmiRNA™ profiling service (Applied Biological Materials Inc.), microRNA Array (Dharmacon—Thermo Scientific), LDA TaqMan analyses (Applied Biosystems), Taqman microRNA Array (Applied Biosystems) or any other commercially available array.

Microarray analysis may comprise all or a subset of the steps of RNA isolation, RNA amplification, reverse transcription, target labelling, hybridisation onto a microarray chip, image analysis and normalisation, and subsequent data analysis; each of these steps may be performed according to a manufacturers protocol.

It follows, that any of the methods as disclosed herein above e.g. for determining the prognosis of an individual with pancreas cancer may further comprise one or more of the steps of:
i) isolating miRNA from a sample,
ii) labelling of said miRNA,
iii) hybridising said labelled miRNA to a microarray comprising miRNA-specific probes to provide a hybridisation profile for the sample,
iv) performing data analysis to obtain a measure of the miRNA expression profile of said sample.

In another embodiment, the microarray for detection of microRNA is custom made.

A probe or hybridization probe is a fragment of DNA or RNA of variable length, which is used to detect in DNA or RNA samples the presence of nucleotide sequences (the target) that are complementary to the sequence in the probe. One example is a sense miRNA sequence in a sample (target) and an antisense miRNA probe. The probe thereby hybridizes to single-stranded nucleic acid (DNA or RNA) whose base sequence allows probe-target base pairing due to complementarity between the probe and target.

To detect hybridization of the probe to its target sequence, the probe or the sample is tagged (or labelled) with a molecular marker. Detection of sequences with moderate or high similarity depends on how stringent the hybridization conditions were applied—high stringency, such as high hybridization temperature and low salt in hybridization buffers, permits only hybridization between nucleic acid sequences that are highly similar, whereas low stringency, such as lower temperature and high salt, allows hybridization when the sequences are less similar. Hybridization probes used in microarrays refer to nucleotide sequences covalently attached to an inert surface, such as coated glass slides, and to which a mobile target is hybridized. Depending on the method the probe may be synthesised via phosphoramidite technology or generated by PCR amplification or cloning (older methods). To design probe sequences, a probe design algorithm may be used to ensure maximum specificity (discerning closely related targets), sensitivity (maximum hybridisation intensities) and normalised melting temperatures for uniform hybridisation.

### Other Analysis Methods

The isolated RNA may be analysed by northern blotting. In one embodiment, the expression level of one or more miRNAs is determined by the northern blot technique.

A northern blot is a method used to check for the presence of a RNA sequence in a sample. Northern blotting combines denaturing agarose gel or polyacrylamide gel electrophoresis for size separation of RNA with methods to transfer the size-separated RNA to a filter membrane for probe hybridization. The hybridization probe may be made from DNA or RNA.

In yet another embodiment, the isolated RNA is analysed by nuclease protection assay.

The isolated RNA may be analysed by Nuclease protection assay.

Nuclease protection assay is a technique used to identify individual RNA molecules in a heterogeneous RNA sample extracted from cells. The technique can identify one or more RNA molecules of known sequence even at low total concentration. The extracted RNA is first mixed with antisense RNA or DNA probes that are complementary to the sequence or sequences of interest and the complementary strands are hybridized to form double-stranded RNA (or a DNA-RNA hybrid). The mixture is then exposed to ribonucleases that specifically cleave only single-stranded RNA but have no activity against double-stranded RNA. When the reaction runs to completion, susceptible RNA regions are degraded to very short oligomers or to individual nucleotides; the surviving RNA fragments are those that were complementary to the added antisense strand and thus contained the sequence of interest.

### Device

It is also an aspect of the present invention to provide a device for measuring the expression level of at least one miRNA in a sample, wherein said device comprises or consists of at least one probe or probe set for at least one miRNA selected from the group consisting of
a) miR-675, miR-212, miR-148a*, miR-187, let-7g*, miR-205, miR-944, miR-431, miR-194*, miR-769-5p, miR-450b-5p, miR-222, miR-146a, miR-23a*, miR-143*, miR-216a, miR-891a, miR-409-5p, miR-449b and miR-330-5p; or
b) miR-675, miR-212, miR-148a*, miR-187 and let-7g*; or
c) miR-675, miR-212, miR-148a*, miR-187, miR-205, miR-944, miR-431, miR-194* and miR-769-5p; or
d) miR-675, miR-212, miR-148a* and miR-187; or
e) miR-675, miR-148a*, miR-450b-5p, miR-222 and miR-146a; or
f) miR-675, miR-148a*, miR-187, miR-205, miR-431, miR-194*, miR-23a*, miR-143*, miR-216a, miR-891a, miR-409-5p, miR-450b-5p, miR-449b and miR-330-5p;
g) miR-675, miR-148a* and miR-450b-5p; or
h) miR-675, miR-187, miR-205, miR-431, miR-194* and 148a*; or
i) miR-675 and miR-148a*; or
j) miR-675, miR-212, miR-148a* and let-7g*; or
k) miR-675, miR-222* and miR-29a*; or
l) miR-148a and miR-625
   wherein said device is used for characterising a sample according to the methods as disclosed herein.

In one embodiment, the device according to the present invention further comprises one or more probes or probe sets for one or more miRNA having previously been identified in the literature as being of prognostic value.

In one embodiment, the device may be used in a method for estimating the probability for a patient with pancreatic cancer of surviving for a certain time period, said method comprising measuring the expression level of at least one miRNA in a sample obtained from said individual.

In one embodiment said device comprises between 1 to 2 probes or probe sets per miRNA to be measured, such as 2 to 3 probes, for example 3 to 4 probes, such as 4 to 5 probes, for example 5 to 6 probes, such as 6 to 7 probes, for example 7 to 8 probes, such as 8 to 9 probes, for example 9 to 10 probes, such as 10 to 15 probes, for example 15 to 20 probes, such as 20 to 25 probes, for example 25 to 30 probes, such as 30 to 40 probes, for example 40 to 50 probes, such as 50 to 60 probes, for example 60 to 70 probes, such as 70 to 80 probes, for example 80 to 90 probes, such as 90 to 100 probes or probe sets per mlRNA of the present invention to be measured.

In another embodiment, said device has of a total of 1 probe or probe set for at least one miRNA to be measured, such as 2 probes, for example 3 probes, such as 4 probes, for example 5 probes, such as 6 probes, for example 7 probes, such as 8 probes, for example 9 probes, such as 10 probes, for example 11 probes, such as 12 probes, for example 13 probes, such as 14 probes, for example 15 probes, such as 16 probes, for example 17 probes, such as 18 probes, for example 19 probes, such as 20 probes, for example 21 probes, such as 22 probes, for example 23 probes, such as 24 probes, for example 25 probes, such as 26 probes, for example 27 probes, such as 28 probes, for example 29 probes, such as 30 probes, for example 31 probes, such as 32 probes, for example 33 probes, such as 34 probes, for example 35 probes, such as 36 probes, for example 37 probes, such as 38 probes, for example 39 probes, such as 40 probes, for example 41 probes, such as 42 probes, for example 43 probes, such as 44 probes, for example 45 probes, such as 46 probes, for example 47 probes, such as 48 probes, for example 49 probes, such as 50 probes or probe sets for at least one miRNA of the present invention to be measured.

It follows, that there may be one probe specific to a miRNA to be measured, or more than one probe specific to a miRNA to be measured—which may be called a probe set. In one embodiment, the device comprises 1 probe per miRNA to be measured, in another embodiment, said device comprises 2 probes, such as 3 probes, for example 4 probes, such as 5 probes, for example 6 probes, such as 7 probes, for example 8 probes, such as 9 probes, for example 10 probes, such as 11 probes, for example 12 probes, such as 13 probes, for example 14 probes, such as 15 probes per miRNA to be measured or analysed.

In one embodiment, the device may be a microarray chip; a QPCR Micro Fluidic Card; or may comprise QPCR tubes, QPCR tubes in a strip or a QPCR plate, comprising one or more probes for at least one miRNA and identified herein.

The probes may be comprised on a solid support, on at least one bead, or in a liquid reagent comprised in a tube.

### Computer Program Product

It is a further aspect of the invention to provide a computer program product having a computer readable medium, said computer program product comprising means for carrying out any of the herein listed methods.

It is a further aspect of the invention to provide a system comprising means for carrying out any of the herein listed methods.

It is an aspect of the present invention to provide a system for estimating the probability for a patient with pancreatic cancer of surviving for a certain time period said system, comprising means for analysing the expression level of at least one miRNA in a sample obtained from an individual with pancreas cancer, wherein said at least one miRNA is selected from the group consisting of
a) miR-675, miR-212, miR-148a*, miR-187, let-7g*, miR-205, miR-944, miR-431, miR-194*, miR-769-5p, miR-450b-5p, miR-222, miR-146a, miR-23a*, miR-143*, miR-216a, miR-891a, miR-409-5p, miR-449b and miR-330-5p; or
b) miR-675, miR-212, miR-148a*, miR-187 and let-7g*; or
c) miR-675, miR-212, miR-148a*, miR-187, miR-205, miR-944, miR-431, miR-194* and miR-769-5p; or
d) miR-675, miR-212, miR-148a* and miR-187; or
e) miR-675, miR-148a*, miR-450b-5p, miR-222 and miR-146a; or
f) miR-675, miR-148a*, miR-187, miR-205, miR-431, miR-194*, miR-23a*, miR-143*, miR-216a, miR-891a, miR-409-5p, miR-450b-5p, miR-449b and miR-330-5p;
g) miR-675, miR-148a* and miR-450b-5p; or
h) miR-675, miR-187, miR-205, miR-431, miR-194* and 148a*; or
i) miR-675 and miR-148a*
j) miR-675, miR-212, miR-148a* and let-7g*; or
k) miR-675, miR-222* and miR-29a*; or
l) miR-148a and miR-625
   In another aspect, the present invention provides a system for performing a prognosis on an individual with pancreas cancer, comprising:
i) means for analysing the miRNA expression profile of a sample obtained from said individual, and
ii) means for estimating the probability for a patient with pancreatic cancer of surviving for a certain time period,
wherein said miRNA expression profile comprises at least one miRNA selected from the group consisting of
a) miR-675, miR-212, miR-148a*, miR-187, let-7g*, miR-205, miR-944, miR-431, miR-194*, miR-769-5p, miR-450b-5p, miR-222, miR-146a, miR-23a*, miR-143*, miR-216a, miR-891a, miR-409-5p, miR-449b and miR-330-5p; or
b) miR-675, miR-212, miR-148a*, miR-187 and let-7g*; or
c) miR-675, miR-212, miR-148a*, miR-187, miR-205, miR-944, miR-431, miR-194* and miR-769-5p; or
d) miR-675, miR-212, miR-148a* and miR-187; or
e) miR-675, miR-148a*, miR-450b-5p, miR-222 and miR-146a; or
f) miR-675, miR-148a*, miR-187, miR-205, miR-431, miR-194*, miR-23a*, miR-143*, miR-216a, miR-891a, miR-409-5p, miR-450b-5p, miR-449b and miR-330-5p;
g) miR-675, miR-148a* and miR-450b-5p; or
h) miR-675, miR-187, miR-205, miR-431, miR-194* and 148a*; or
i) miR-675 and miR-148a*
j) miR-675, miR-212, miR-148a* and let-7g*; or
k) miR-675, miR-222* and miR-29a*; or
l) miR-148a and miR-625

In another aspect, the present invention provides a computer program product having a computer readable medium, said computer program product providing a system for estimating the prognosis of an individual with pancreas cancer, said computer program product comprising means for carrying out any of the steps of any of the methods as disclosed herein.

In another aspect, the present invention provides a system as disclosed herein wherein the data is stored, such as stored in at least one database.

### Kit-of-Parts

It is also an aspect to provide a kit-of-parts comprising the device according to the present invention, and at least one additional component.

In one embodiment, the additional component may be used simultaneously, sequentially or separately with the device.

In one embodiment, said additional component comprises means for extracting RNA such as miRNA from a sample; reagents for performing microarray analysis and/or reagents for performing QPCR analysis.

In another embodiment, said kit may comprise instructions for use of the device and/or the additional components.

In a further embodiment, said kit comprises a computer program product having a computer readable medium as detailed herein elsewhere.

Sequences

| miR name | Alias (ID) | Accession no. | Sequence |
|---|---|---|---|
| hsa-let7g* | hsa-let-7g-3p | MIMAT0004584 | cuguacaggccacugccuugc |
| hsa-miR-675 | hsa-miR-675-5p | MIMAT0004284 | uggugcggagagggcccacagug |
| hsa-miR-212 | hsa-miR-212-3p | MIMAT0000269 | uaacagucuccagucacggcc |
| hsa-miR-148a* | miR-148a-5p | MIMAT0004549 | aaaguucugagacacuccgacu |
| hsa-miR-187 | hsa-miR-187-3p | MIMAT0000262 | ucgugucuuguguugcagccgg |
| hsa-miR-205 | hsa-miR-205-5p | MIMAT0000266 | uccuucauuccaccggagucug |
| hsa-miR-944 | — | MIMAT0004987 | aaauuauuguacaucggaugag |
| hsa-miR-431 | hsa-miR-431-5p | MIMAT0001625 | ugucuugcaggccgucaugca |
| hsa-miR-194* | hsa-miR-194-3p | MIMAT0004671 | ccaguggggcugcuguuaucug |
| hsa-miR-769-5p | — | MIMAT0003886 | ugagaccucuggguucugagcu |
| hsa-miR-450b-5p | — | MIMAT0004909 | uuuugcaauauguuccugaaua |
| hsa-miR-222 | hsa-miR-222-3p | MIMAT0000279 | agcuacaucuggcuacugggu |
| hsa-miR-146a | hsa-miR-146a-5p | MIMAT0000449 | ugagaacugaauuccauggguu |
| hsa-miR-23a* | hsa-miR-23a-5p | MIMAT0004496 | gggguuccuggggaugggauuu |
| hsa-miR-143* | hsa-miR-143-5p | MIMAT0004599 | ggugcagugcugcaucucuggu |
| hsa-miR-216a | hsa-miR-216 | MIMAT0000273 | uaaucucagcuggcaacuguga |
| hsa-miR-891a | — | MIMAT0004902 | ugcaacgaaccugagccacuga |
| hsa-miR-409-5p | — | MIMAT0001638 | agguuacccgagcaacuuugcau |
| hsa-miR-449b | hsa-miR-449b-5p | MIMAT0003327 | aggcaguguauuguuagcuggc |
| hsa-miR-330-5p | — | MIMAT0004693 | ucucugggccugugucuuaggc |
| hsa-miR-222* | hsa-miR-222-5p | MIMAT0004569 | cucaguagccaguguagauccu |
| hsa-miR-29a* | hsa-miR-29a-5p | MIMAT0004503 | acugauuucuuuugguguucag |
| hsa-miR-148a | hsa-miR-148a-3p | MIMAT0000243 | ucagugcacuacagaacuuugu |
| hsa-miR-625* | hsa-miR-625-3p | MIMAT0004808 | gacuauagaacuuucccccuca |

### Example 1

### Prognostic microRNAs in Tissue from Patients Operated for Pancreatic Cancer

### Abstract

### Purpose

The aim was to identify a panel of microRNAs that can predict overall survival (OS) in non-microdissected cancer tissues from patients operated for pancreatic cancer (PC).

### Patients and Methods

MiRs were purified from cancer tissue from 225 patients operated for PC. Expressions of microRNAs were determined using TaqMan® MicroRNA Array v2.0 (ABI). Univariate selection and the Lasso method were applied before the Cox proportional hazard model to relate miRs to OS.

### Results

High expression of miR-212 (HR 1.54, Cl 1.21-1.96) and miR-675 (1.08, 1.02-1.14), and low expression of miR-148a* (0.92, 0.88-0.98), miR-187 (0.97, 0.94-1.00) and let-7g* (0.83, 0.73-0.95) predicted short OS independent of age, gender, calendar year of operation, KRAS mutation status, tumor stage, ASA-score, localization and differentiation of tumor. A nomogram based on the adjusted Cox proportional hazard model was determined for these microRNAs and a prognostic index (PI) for each patient was calculated. The median survival was 1.09 years (Cl 0.98-1.43) when PI was >median PI compared to 2.23 years (Cl 1.84-4.36) when PI was <median. MiR-212, miR-675, miR-187, miR-205, miR-944, miR-431, miR-194*, miR-148a*, and miR-769-5p showed the strongest prediction ability by the Lasso method. Thus miR-212, miR-675, miR-187 and miR-148a* were predictors for OS in both statistical methods. High expression of miR-675, miR-450b-5p and miR-222, and low expression of miR-148a* and miR-146a could predict short 2 years OS independent of clinical characteristics.

### Conclusion

The combination of the expression of 5 microRNAs can identify PC patients with short overall survival after radical surgery. Patients with a prognostic index>median had a very short OS. These results are independent of chemotherapy.

### Introduction

Pancreatic cancer (PC) is the 4^{th} most common cause of cancer death in Western World. The prognosis of patients with PC is dismal, and only 5% are alive after 5 years (1). The poor prognosis of patients with PC is a results of late clinical presentation, early and high metastatic potential and resistance to chemotherapy and radiation. Most of PCs are ductal adenocarcinomas (PDAC). Less than 20% of these patients can be operated with curative intent, and the 5-years survival after curative intended surgery is 20% (2, 3). Approximately 12% of periampullary tumors are ampullary adenocarcinomas (A-AC), which have 45% 5-years survival after curative intended surgery (4, 5). No biomarkers can today reliable predict the prognosis of patients with PC operated with curative intent (6).

MicroRNAs are 17-25 nucleotide-long non-coding RNAs which regulate gene expression posttranscriptionally by a binding of specific mRNA. MicroRNAs play essential roles in basic biological functions such as tumor growth, invasion, angiogenesis, proliferation, differentiation, and regulate epithelial-mesenchymal transition (EMT) and cancer stem cells (7-11). miRNAs are stable in formalin-fixed embedden (FFPE) tissue, which is suitable for miRNA analysis. More than 1500 human microRNAs sequences are described to date (http://www.mirbase.org/index.shtml, November 2011).

The expression of microRNAs in tissue and blood are emerging as new diagnostic, predictive and prognostic biomarkers in patients with PC (12-26). In PC tissue some microRNAs are up-regulated and some down-regulated compared to normal pancreas tissue, and some act as oncogene or tumor supressor genes. The miR expression profiles of tissue from PC and A-AC are almost identical and few miRs are differently expressed between the two cancers (27). Different expression in PC tissue or plasma of let-7d, miR-21, miR-125a, miR-142-5p, miR-152, miR-155, miR-181c, miR-196a-2, miR-203, miR-204 miR-210, miR-222, and miR-518b, are associated to prognosis in patients with PC (13, 15, 21-26).

The aim of the present study was to identify new prognostic microRNAs in PC tissue, without micro-dissection, from patients operated with curative intentions for PC, and to combine miRNAs in a prognostic model.

### Patients and Methods

The study was conducted according to the REMARK guidelines (28).

### Patients and Inclusion Criteria

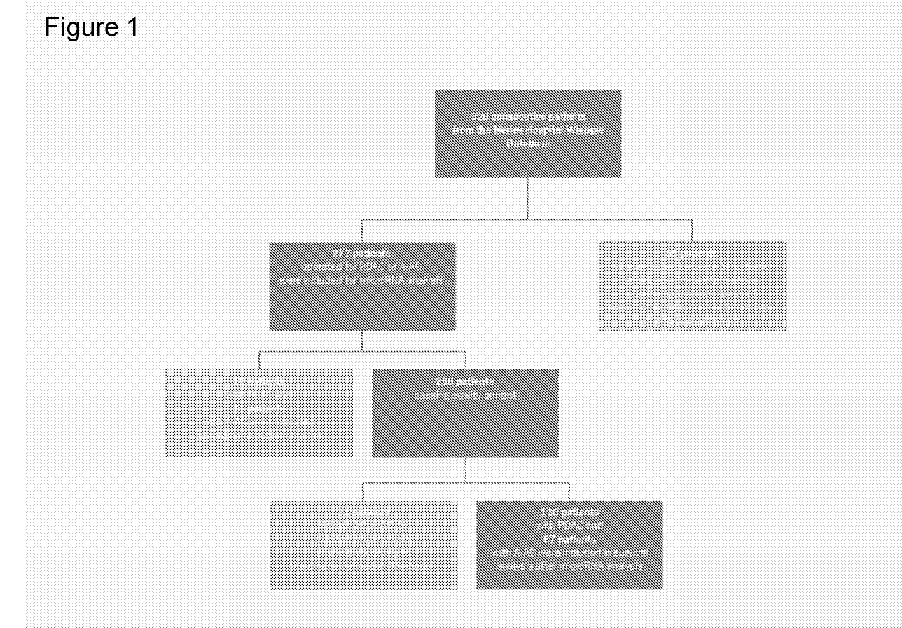

328 consecutive patients were operated with radical intentions for lesions in the pancreas at Herlev Hospital between December 1976 and June 2008. Roughly 99% were Caucasians of Danish descent. Archival formalin fixed paraffin embedded (FFPE) blocks were available from 322 patients. New sections from 5-10 tissue blocks, representing tumor and normal pancreas, from each patient were stained with hematoxylin and eosin (HE) and examined by two experienced pathologists. All tumors were classified and graded according to WHO criteria (29). An existing database with clinical information was updated. 170 patients had pancreatic adenocarcinomas of ductal origin (PDAC) and 107 had A-AC. Excluded were patients where tissue blocks were missing and 36 patients with chronic pancreatitis, non-invasive tumor or tumor of non-ductal origin. 257 patients underwent a pancreaticoduodenectomy (4), 13 a distal pancreatectomy, and 7 a total pancreatectomy. Thus 277 patients were included in the microRNA analysis. Patient selection is illustrated in the CONSORT diagram (FIG. 1). Patients were followed from their date of operation until death, emigration (one patient) or censoring Oct. 13, 2010, whichever came first. Thirty-two (14%) were treated with gemcitabine, 5-FU or cisplatin as adjuvant and/or palliative chemotherapy. The study was approved by the local Ethical Committee (protocol H-KA-20060181). The Danish Registry of Human Tissue Utilization was consulted.

### MicroRNA Analysis

FFPE blocks representing tumor tissue were selected from each patient for microRNA analysis. Three 10 μm sections were cut from each of the FFPE samples for microRNA extraction and placed in a sterile eppendorf tube. Since the PC tissue was not micro-dissected, the microRNAs will originate from both cancer cells and stroma cells. MicroRNA was extracted from the FFPE tissue using High Pure miRNA Isolation Kit (Roche) according to the manufactures' instructions. In brief, the tissue sections were deparaffinized in xylene and ethanol, then treated with proteinase K and finally RNA was isolated using the one-column spin column protocol for total RNA. The concentration of RNA was assessed by absorbance spectrometry on NanoDrop X-1000 (Thermo Fisher Scientific, Inc.). The microRNA profiling was performed on TaqMan® Array Human MicroRNA A+B Cards v2.0 (Applied Biosystems) using the manufactures reagents and instructions. Each array analyzes 664 different human microRNAs and enables a comprehensive expression profile consistent with Sanger miRBase v14 (human). Briefly, the RNA was transcribed into cDNA in two multiplex reactions each containing 200 ng of RNA and either Megaplex RT Primer A Pool or B pool and using the TaqMan MicroRNA Reverse Transcription Kit in a total volume of 14 μl. Prior to loading the 12 cycle preamplification reaction was performed using 2.5 μl cDNA in a 25 μl reaction. Each of the arrays was loaded with 800 μl Universal PCR MasterMix assay containing 1/40 of the preamplification reaction and run on the 7900HT Fast Real-Time PCR System. Samples were analyzed at AROS Applied Biotechnology A/S, Aarhus, Denmark.

### Statistical Analysis

Raw Ct values where pre-processed in the following steps: 1) missing values and Ct values above 32 were flagged: 2) repeat measurements (excluding flagged values) where averaged; 3) features that were flagged in more than a given percent of samples were removed from the dataset; 4) missing values were set to Ct=40; and 5) quantile normalization was performed (30). For QC of samples, the threshold in step 3 was set to 80%. Normalized data was inspected for outliers and potential technical bias from sample quality, sample purification date and TLDA array batch. No heavy technical bias was observed. However, 21 samples were identified as outliers. Most samples' Ct density curves were bimodal with peaks around 29 and 40. In some cases, the peak around 40 was relatively high compared to the peak around 29 and these samples corresponded well to outliers identified by principal component analysis. We therefore removed samples from the dataset if the ratio between the peaks at Ct>32 vs. Ct<32 was above 0.9 (outlier criteria 1:density ratio>0.9) or if their average correlation (Pearson correlation) with other samples in the dataset was below 0.70 (outlier criteria 2:average correlation<0.7). Furthermore, samples that were close to failing both criteria were also categorized as outliers (outlier criteria 3:density ratio>0.8 and average correlation<0.77). Samples that passed QC were pre-processed as described above with the threshold in step 3 now set to 95%.

We applied the Cox proportional hazard model in order to study the effect of microRNA expression on OS. Since we had far more microRNA expression values than patients, the parameter estimates were obtained by dimension reduction methods. We applied two different methods: the univariate selection method (31) and the Lasso method (31, 32), in order to check whether different methods would yield same results. The univariate selection method implies testing of each microRNA expression value on survival. This was done by fitting the univariate Cox proportional hazard model and testing each microRNA separately. All microRNA that met the 0.0001 significance level (approximately 0.05/number of tests; Bonferroni adjustment) in the univariate analysis was then kept and included in a multivariate Cox proportional hazard model. The final model was obtained by backwards elimination of the multivariate Cox proportional hazard model and applying a significance level of 5%.

Significant microRNA expressions in the final model were presented in terms of hazard ratios (HR), per unit increase, 95% confidence intervals (CI), and p-values. All results were presented with and without adjustment for age, gender, calendar time, ASA-score (physical status classification system, American Society of Anaesthesiologists), tumor differentiation and location, stage and KRAS mutation status. Test of the proportional hazards assumption was based on weighted residuals (33). The Lasso method is primarily used for prediction. It shrinks the regression coefficients towards zero by penalizing the size of the coefficients using the L₁ norm, i.e. the absolute value of the coefficients (32). Penalizing with the absolute value has the effect that a number of the estimated coefficients will become zero, and thus the Lasso method can be used as a selection method (31). The tuning parameter which determines the shrinkage was determined by means of 10-fold cross-validation. Most significant microRNAs in the Lasso model are listed without HR and p-values. Calculations were repeated with only two years follow-up; i.e. all survival times above 24 months are censored in the analysis.

We calculated the prognostic index (PI) for each patient by multiplying each of the significant microRNA expressions (xᵢ) with their corresponding estimated coefficient (βᵢ) from the cox regression and summing over all terms, i.e. P.I.=Σ_{ι}xᵢβᵢ. We then divided the patients into two groups, those with PI below the median PI and those with PI above the median PI.

The statistical software R (34) version 2.10.1 was used in all analysis. We used the package survival version 2.35-7 for fitting the Cox proportional hazard model, the package penalized version 0.9-31 for obtaining the estimates by the Lasso procedure, and the package Design version 2.3-0 for creating the nomograms.

Excluded in survival analysis were: 1) patients who died of complications to surgery during the first 30 days of operations and patients who died of complications to surgery and never left the hospital (n=23); 2) patients who also had another cancer diagnosis, and where the cause of death is not clear (n=3); and 3) patients operated with a R2 resection (n=5). Patients operated with a R1 resection are included in the study (n=7).

### Results

Clinical Characteristics of the Patients with PC Included in the Analysis of Prognostic microRNAs

RNA extraction was satisfying in all 277 PC samples (mean 260/280 nm absorbance ratio was 1.85). After microRNA profiling ten (6%) PDAC samples and eleven (10%) A-AC samples were excluded according to the defined normalization criteria. 256 samples (PDAC n=160, A-AC n=96) were pre-processed as described resulting in a pre-processed dataset comprising 256 samples and 423 microRNAs for analysis. Outliers were distributed between tumor blocks of all ages, and no clustering related to the age of the tumor blocks was found. 31 patients were excluded according to the clinical criteria. FIG. 1 illustrates the consort diagram. Table 1 gives baseline characteristics of the 225 PC patients included in the statistical analysis.

Association of microRNAs with OS

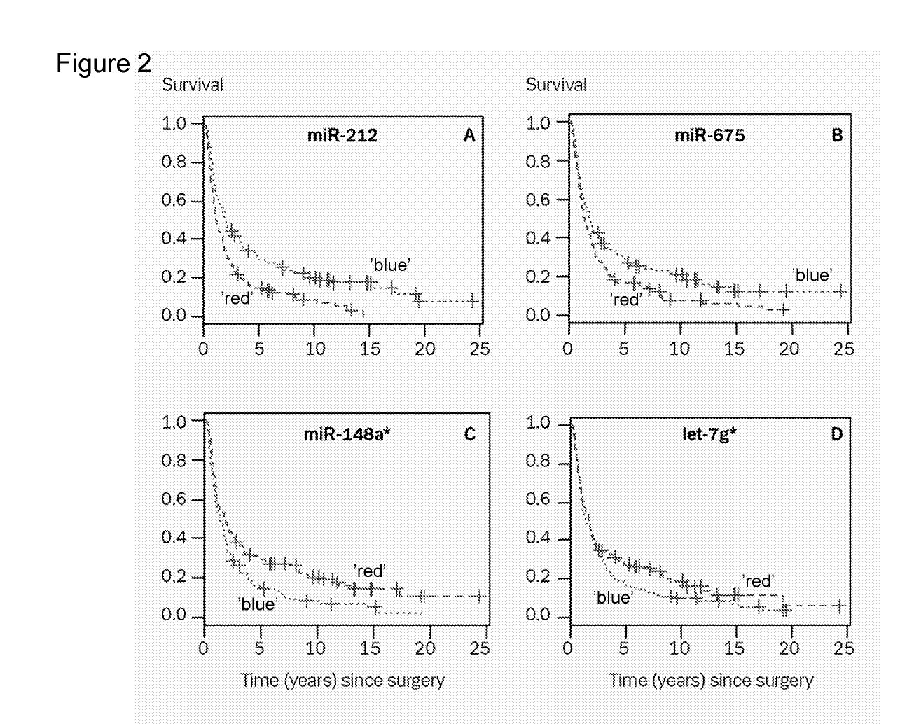

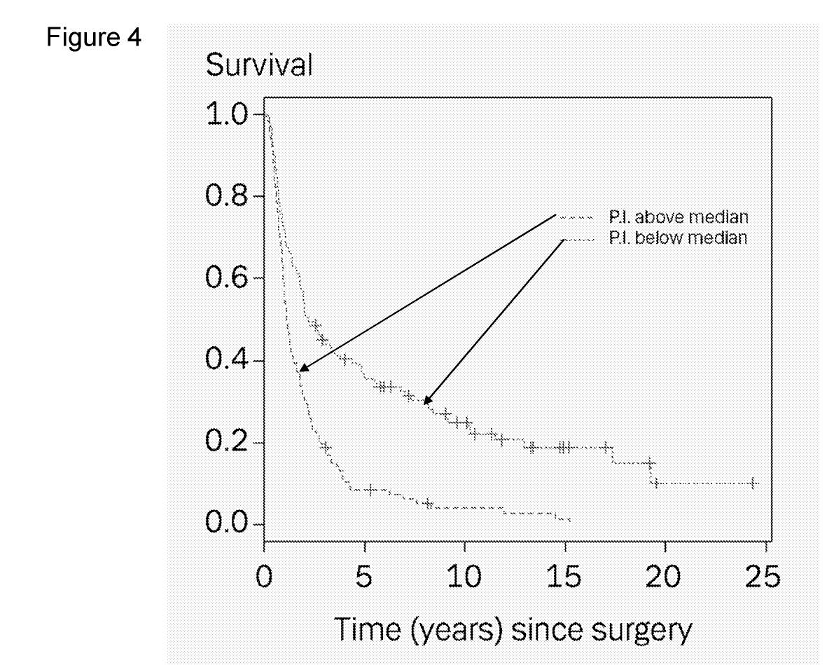

At time of follow-up 196 (87%) patients were dead. Shortest survival time was 38 days. In the survival analysis of 225 patients with PC and using the Cox proportional hazard model with backwards elimination the following microRNAs were prognostic for OS: miR-212, miR-675, miR-148a*, miR-187 and let-7g*. The hazard ratios, calculated per unit increase for the 40-Ct value of these microRNAs, are shown in Table 2 (top-left). High expression of miR-212 and miR-675, and low expression of miR-148*, miR-187 and let-7g* predicted short OS independent of age, gender, calendar year of operation, KRAS mutation status, tumor stage, ASA-score, localization and differentiation of tumor (Table 2, top-right). In the adjusted analysis KRAS (p=0.030), tumor stage (p<0.001), ASA-score (p=0.013), and tumor differentiation (p<0.001) were associated with OS, whereas gender, year of surgery and localisation were not significant. FIG. 2 (A-D) illustrates the Kaplan-Meier survival curves for the four most significant microRNAs. FIG. 3 illustrates a nomogram based on the adjusted Cox proportional hazard model. This nomogram predicts 3 months, 1 year and 10 years OS probability in patients with PC operated with radical intention according to expressions of miR-212, miR-675, miR-148a*, miR-187 and let-7g*. Based on the microRNAs in the Cox proportional hazard model we calculated a prognostic index (PI) for each patient. FIG. 4 illustrates the Kaplan-Meier survival curves for patients with PI above and below median PI. The median survival was 1.09 years (CI: 0.98-1.43) when PI was >median PI compared to 2.23 years (CI: 1.84-4.36) when PI was <median PI.

The nine microRNAs with strongest prediction ability by the Lasso method, when sorted by size of effect, were miR-212, miR-675, miR-187, miR-205, miR-944, miR-431, miR-194*, miR-148a*, and miR-769-5p (Table 3, left). Thus miR-212, miR-675, miR-187 and miR-148a* were identified as predictors for OS in both statistical methods.

Association of microRNAs with 2 Years OS

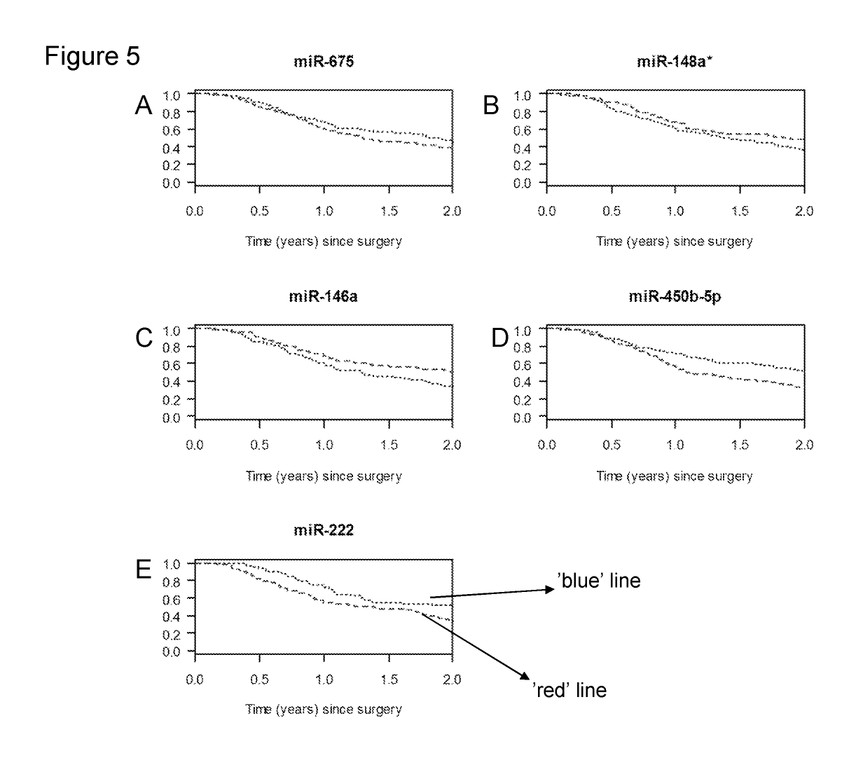

We repeated the Cox proportional hazard model (Table 2, bottom) and the Lasso-model (Table 3, right) with only 2 years follow-up. The following microRNAs were prognostic for 2 years follow-up: miR-675, miR-148a*, miR-146a, miR-450b-5p and miR-222. The hazard ratios, calculated per unit increase for the 40-Ct value, of these miRs are shown in Table 2 (bottom, left). High expression of miR-675, miR-450b-5p and miR-222, and low expression of miR-148a* and 146a predicted short 2 years survival independent of age, gender, calendar year of operation, KRAS mutation status, stage, ASA-score, localization and differentiation of tumor (Table 2, bottom, right). In the adjusted analysis tumor stage (p<0.001), ASA-score (p=0.008), and tumor differentiation (p<0.001) were associated with 2 years survival, whereas gender, KRAS mutation status, year of surgery and localisation were not significant. FIG. 5 (A-E) illustrates the Kaplan-Meier survival curves for the five most significant microRNAs. The 14 microRNAs with strongest prediction ability by the Lasso method are given in Table 3 (right). MiR-675, miR-148a*, and miR-450b-5p were identified as predictors for 2 years survival in both statistical methods.

MiR-675, miR-187, miR-205, miR-431, miR-194* and miR-148a* were predictors for both OS and 2 years survival calculated by the Lasso method.

Association of miRNAs with OS for PDAC

If calculations for OS were done only for PDAC, adjusted analysis showed that high expression of miR-675 (HR=1.40, P=0.026) and miR-222* (HR=1.57, P=0.003), and low expression of miR-29a* (HR=0.70, P=0.003) predicted short OS independent of age, gender, calendar year of operation, chemotherapy, KRAS mutation status, tumor stage, ASA-score and differentiation of tumor.

Association of miRNAs with OS for A-AC

If calculations for OS were done only for A-AC, adjusted analysis showed that low expression of miR-148a (HR=0.45, P=0.001) and miR-625 (HR=0.54, P<0.001) predicted short OS independent of age, gender, calendar year of operation, chemotherapy, KRAS mutation status, tumor stage, ASA-score and differentiation of tumor.

Association of Prognostic microRNAs Described in the Literature

The following microRNAs were tested without Bonferroni adjustment since they are reported as prognostic biomarkers in PC patients: High expression of miR-21 (HR 1.16, Cl 0.974-1.385, p=0.09) and miR-203 (1.18, 1.08-1.30, p=0.0005), and low expression of miR-10b (0.87, 0.73-1.03, p=0.09) miR-196b (0.89, 0.83-0.96 p=0.002), miR-222 (0.74, 0.61-0.90, p=0.0023) and let-7d (0.83, 0.69-1.00, p=0.051) were predictors of short OS. miR-142-5p (p=0.96), miR-152 (p=0.39), miR-181c (p=0.64), miR-204 (p=0.29), miR-210 (0.99, p=0.90) and miR-518b (p=0.22) were not associated with OS. MiR-155 was not present in our microRNAarray and miR-155* variant was removed in data pre-processing.

MiR-148a has tumor suppressor potential in PC and comes from the same transcript and miR-precursor microRNA as miR-148a*. These two microRNAs had very similar expression profiles in our study (28). Univariate analysis demonstrated that low expression of miR-148a (0.89, 0.81-0.97, p=0.012) and miR-148a* (0.89, 0.84-0.94, p<0.0001) were predictors of short OS.

When miR-34a was tested in survival analysis with the median value as cut-off as described by Jamieson et al., we found that miR-34a was associated with poor OS; miR-34a (HR=0.64, 0.44-0.92, p=0.018). We found a tendency to association between high expression of miR-21 and short OS(HR 1.16, Cl 0.97-1.39, p=0.09)

### DISCUSSION

Only 20% of patients operated for PC with curative intentions are alive after 5-years. Better biomarkers to identify patients with poorest prognosis are needed in order to treat them more aggressively and are thus valuable in the selection of the right treatment algorithm. Pancreatic cancer is characterized by large amount of connective tissue surrounding the cancer cells. Our large retrospective study was therefore conducted to identify microRNAs in PC tissue, including cancer cells and stroma, which could predict OS in patients with PC operated with curative intentions. Few patients had received postoperative gemcitabine—thus, one other strength of our study was that most of the patients did not receive post-operative chemotherapy. The identified miRNAs therefore reflect the effect of operation.

We decided to calculate one prognostic index for both PDAC and A-AC (collectively PC). Pancreatic cancer cells and ampullary cancer cells, of both pancreato-biliary and intestinal subtypes, originate from the same stem cell. And in a previous study of expression profiles we found that PDAC and A-AC have almost identical expression profiles when compared to each other and normal pancreas, chronic pancreatitis and duodenal adenocarcinomas. The population size (n) is of large importance in array studies, and after Bonferoni adjustment and adjusted analysis including tumor localization (PDAC or A-AC) this method is strong and give reliable results.

In the adjusted analysis we found that high expression of miR-212 and miR-675, and low expression of miR-148a*, miR-187 and let-7g* predicted short OS after operation for PC, and were independent of age, gender, calendar year of operation, KRAS mutation status, stage, ASA-score, localization and differentiation of tumor. MiR-212, miR-675, miR-187 and miR-148a* were predictors for OS in both statistical approaches. We also combined the expression of these microRNAs with clinical characteristics in a prognostic index that provided a strong prediction of OS for patients operated for PC. This is novel observations and a new way of combining prognostic miRNAs.

When the follow-up period was only 2 years, high expression of miR-675 and low expression of miR-148a* remained significant, and 3 new microRNAs were identified, miR-146a, miR-222 and miR-450b-5p. If Bonferroni adjustment was excluded most of the prognostic microRNAs described in literature were validated (15, 21-26).

Serum CA 19-9 is the most used biomarker of OS in patients with locally advanced and metastatic PC (2, 6). Peri-operative serum CA 19-9 is also an independent biomarker of OS (35, 36). A limitation of serum CA 19-9 is that 10% of patients with PC do not produce CA 19-9 even with advanced disease (2). Serum CA 19-9 was not determined in the present study. In an ongoing prospective study of patients with PC we will include our prognostic index based on microRNA expression in PC tissue, serum CA 19-9 and clinical characteristics.

Low expression of let-7g* was the most significant predictor of short OS in the multivariate analysis of OS, and the nomogram showed that changes in let-7g* expression had high impact on survival. In a recent study of 31 patients with PC at different stages the expression of let-7b and let-7d in pre-treatment plasma samples were low and inversely related with survival (24). Let-7 inhibits cell proliferation and KRAS signalling and is reduced in tissue from PDAC (37). Downregulation of let-7b, let-7c, let-7d and let-7e are related to cetuximab resistance and activation of signalling downstream to KRAS in patients with colorectal cancer (38, 39).

Others have reported similar expression of miR-148a (38). Mir-148a* and miR-148a come from same gene transcript and microRNA-precursor, and both are significantly down-regulated in tissue from PC and A-AC compared to normal pancreas (13, 14). Hypermethylation of the encoding DNA region is responsible for the down-regulation of miR-148a, and this microRNA is an early biomarker of PC since it is already decreased in pre-neoplastic PanIN lesions (40). Twenty-seven target genes are known for miR-148a, including, cell division cycle 25 B (41), bladder cancer associated protein, RAB34 member RAS oncogene family and stromal cell derived factor receptor 1 (42). MiR-148a is also related to lymph node metastasis in gastric cancer (43).

We have found a significantly down-regulation of miR-148a in KRAS mutated PDAC compared to wild type tumors. KRAS mutations are more common in PDAC than in A-AC [42] and KRAS mutations were found in 80% of the included PDAC samples. This explains why miR-148a* in PDAC was significantly associated with OS in the unadjusted analysis but not in the adjusted analysis and why we decided to keep miR-148a* in the prognostic index.

MiR-187 and miR-212 are related to RAS or EGFR signalling (44, 45). High expression of miR-675 was associated with short OS in the present study. H19 is the precursor to miR-675, and the tumoriogenic process induced by H19 may be mediated through miR-675 (46). H19 is transcribed from maternally expressed oncofetal gene located on chromosome 11p15.5, and is over-expressed from the early stages of embryogenesis to fetal life in many organs including fetal liver and placenta. H19 expression is up-regulated in many cancers including colorectal-, hepatocellular-, esophageal-, testicular-, ovarian-, breast- and chorio-carcinoma. MiR-675 is over-expressed in AFP-secreting HCC-cell lines compared to non-secreting (46, 47).

In conclusion, high expressions of miR-212 and miR-675 and low expression of miR-148a*, miR-187 and let-7g* in non-microdissected PC tissue were independent predictors of short OS in a large cohort of patients operated for PC (PDAC and A-AC). We have described a prognostic index based on these microRNAs and clinical characteristics, which can identify patients with short survival after operation. The results demonstrated that microRNAs in PC tissue have the potential as new prognostic biomarkers in patients operated for PC.

### REFERENCES

1. Jemal A, Siegel R, Xu J, et al: Cancer statistics, 2010. CA Cancer J Clin 60:277-300, 2010
2. Hildago M: Pancreatic cancer. N Engl J Med 362:1605-1612, 2010
3. Wagner M, Redaelli C, Lietz M, et al: Curative resection is the single most important factor determining outcome in patients with pancreatic adenocarcinoma. Br J Surg 91:586-594, 2004
4. Beger H G, Thorab F C, Liu Z, et al: Pathogenesis and treatment of neoplastic diseases of the papilla of Vater: Kausch-Whipple procedure with lymph node dissection in cancer of the papilla of Vater. J Hepatobiliary Pancreat Surg 11:232-238, 2004
5. Bettschart V, Rahman M Q, Engelken F J, et al: Presentation, treatment and outcome in patients with ampullary tumours. Br J Surg 91:1600-1607, 2004
6. Locker G Y, Hamilton S, Harris J, et al: ASCO 2006 update of recommendations for the use of tumor markers in gastrointestinal cancer. J Clin Oncol 24:5313-5327, 2006
7. Nelson K M, Weiss G J: MicroRNAs and cancer: past, present and potential future. Mol Cancer Ther 7:3655-3660, 2008
8. Bartels C L, Tsongalis G J: MicroRNAs: novel biomarkers for human cancer. Clin Chem 55:623-631, 2009.
9. lorio M V, Croce C M: MicroRNAs in cancer: small molecules with a huge impact. Clin Oncol 27:5848-5856, 2009
10. Visone R, Croce M: MiRNAs and cancer. Am J Pathol 174:1131-1138, 2009.
11. Farazi T A, Spitzer J I, Morozov P, et al: miRNAs in human cancer. J Pathol 223:102-115, 2011
12. Rachagani S, Kumar S, Batra S K: MicroRNA in pancreatic cancer: pathological, diagnostic and therapeutic implications. Cancer Lett 292:8-16, 2010
13. Bloomston M, Frankel W L, Petrocca F, et al: MicroRNA expression patterns to differentiate pancreatic adenocarcinoma from normal pancreas and chronic pancreatitis. JAMA 2007; 297:1901-1908, 2007
14. Szafranska A E, Davison T S, John J, et al: MicroRNA expression alterations are linked to tumorigenesis and non-neoplastic processes in pancreatic ductal adenocarcinoma. Oncogene 26:4442-4452, 2007
15. Dillhoff M, Liu J, Frankel W, et al: MicroRNA-21 is overexpressed in pancreatic cancer and a potential predictor of survival. J Gastrointest Surg 12:2171-2176, 2008
16. Szafranska A E, Doleshal M, Edmunds H S, et al: Analysis of microRNAs in pancreatic fine-needle aspirates can classify benign and malignant tissues. Clin Chem 54:1716-1724, 2008
17. Habbe N, Koorstra J-B M, Mendell J T, et al: MicroRNA miR-155 is a biomarker of early pancreatic neoplasia. Cancer Biol Ther 8:340-346, 2009
18. Seux M, lovanna J, Dagorn J-C, et al: MicroRNAs in pancreatic ductal adenocarcinoma: new diagnostic and therapeutic clues. Pancreatology 9:66-72, 2009
19. Wang J, Chen J, Chang P, et al: MicroRNAs in plasma of pancreatic ductal adenocarcinoma patients as novel blood-based biomarkers of disease. Cancer Prev Res 2:807-813, 2009
20. Zhang Y, Li M, Wang H, et al: Profiling of 95 microRNAs in pancreatic cancer cell lines and surgical specimens by real-time PCR analysis. World J Surg 33:698-709, 2009
21. Greither T, Grochola L F, Udelnow A, et al: Elevated expression of microRNAs 155, 203, 210 and 222 in pancreatic tumors is associated with poorer survival. Int J Cancer 126:73-80, 2010
22. Hwang J H, Voortman J, Giovanneti E, et al: Identification of microRNA-21 as a biomarker for chemoresistance and clinical outcome following adjuvant therapy in resectable pancreatic cancer. PLoS One 5:e10630, 2010
23. Ikenaga N, Ohuchida K, Mizumoto K, et al: MicroRNA-203 expression as a new prognostic marker of pancreatic adenocarcinoma. Ann Surg Oncol 17:3120-3128, 2010
24. Ali S, Almhanna K, Chen W, et al: Differential expressed miRNAs in the plasma may provide a molecular signature for aggressive pancreatic cancer. Am J Transl Res 3:28-47, 2011
25. Kong X, Du Y, Wang G, et al: Detection of differentially expressed microRNAs in serum of pancreatic ductal adenocarcinoma patients: miR-196a could be a potential marker for poor prognosis. Dig Dis Sci 56:602-609, 2011
26. Ohuchida K, Mizumoto K, Kayashima T, et al: MicroRNA expression as a predictive marker for gemcitabine response after surgical resection of pancreatic cancer. Ann Surg Oncol, 2011 Feb. 23, Epub ahead of print
27. Schultz N A, Werner J, Willenbrock H, et al: MicroRNA expression profiles associated with pancreatic cancer. J Clin Oncol 29: supplement 4: abstract 153, 2011
28. McShane L M, Altman D G, Sauerbrei W, et al: Reporting recommendations for tumor MARKer prognostic studies (REMARK). Breast Cancer Res Treat 100:229-235, 2006
29. WHO Classification of Tumours. Pathology and genetics of tumours of the digestive system. Lyon: IARC Press; 2000
30. Mar J C, Kimura, Schroder K, et al: Data-driven normalization strategies for high-throughput quantitative RT-PCR. BMC Bioinformatics 10:110, 2009
31. Bovelstad H M, Nygard S, Storvold H L, et al: Predicting survival from microarray data—a comparative study. Bioinformatics 23:2080-2087, 2007
32. Tibshirani R: The lasso method for variable selection in the Cox model. Stat Med 16:385-395, 1997
33. Grambsch P, Therneau T: Proportional hazards tests and diagnostics based on weighted residuals. Biometrika 81:515-526, 1994
34. R Development Core Team. R: A Language and environment for statistical computing. R Foundation for Statistical Computing: Vienna, Austria, 2007. ISBN 3-900051-07-0
35. Ferrone C R, Finkelstein D M, Thayer S P, et al: Perioperative CA 19-9 levels can predict stage and survival in patients with respectable pancreatic adenocarcinoma. J Clin Oncol 24:2897-2902, 2006
36. Smith R A, Bosonnet L, Ghaneh P, et al: Preoperative CA 19-9 levels and lymph node ratio are independent predictors of survival in patients with resected pancreatic ductal adenocarcinoma. Dig Surg 25:226-232, 2008
37. Torrisani J, Bournet B, du Rieu M C, et al: Let-7 microRNA transfer in pancreatic cancer derived cells inhibits in vitro cell proliferation but fails to alter tumor progression. Hum Gen Ther 20:831-844, 2009
38. Johnson S M, Grosshans H, Shingara J, et al: RAS is regulated by the let-7 microRNA family. Cell 120:635-647, 2005
39. Ragusa M, Majorana A, Statello L, et al: Specific alterations of microRNA transcriptome and global network structure in colorectal carcinoma after cetuximab treatment. Mol Cancer Ther 9:3396-3409, 2010
40. Hanoun N, Delpu Y, Suriawinata A A, et al: The silencing of microRNA 148a production by DNA hypermethylation is an early event in pancreatic carcinogenesis. Clin Chem 56:1107-1118, 2010
41. Grutzmann R, Foerder M, Alldinger I, et al: Gene expression profiles of microdissected pancreatic ductal adenocarcinoma. Virchows Arch 443:508-517, 2003
42. Buchholz M, Braun M, Heidenblut A, et al: Transcriptome analysis of microdissected pancreatic intraepithelial neoplastic lesions. Oncogene 24:6626-6636, 2005
43. Tchernitsa O, Kasajima A, Schafer R, et al: Systematic evaluation of the miRNA-ome and its downstream effects on mRNA expression identifies gastric cancer progression. J Pathol 222:310-319, 2010
44. Nikiforova M N, Tseng G C, Steward D, et al: MicroRNA expression profiling of thyroid tumors: biological significance and diagnostic utility. J Clin Endocrinol Metab 93:1600-1608, 2008
45. Hatakeyama H, Cheng H, Wirth P, et al: Regulation of heparin-binding EGF-like growth factor by miR-212 and acquired cetuximab-resistance in head and neck squamous cell carcinoma. PLoS Once 5:e12702, 2010
46. Tsang W P, Ng E K, Ng S S, et al: Oncofetal H19-derived miR-675 regulates tumor suppressor R B in human colorectal cancer. Carcinogenesis 31:350-358, 2010
47. Hernandez J M, Schibata D, Bloom G C, et al: Expression of miR-675 in alpha-fetoprotein-secreting hepatocellular carcinoma. ASCOGI 2010, abstract 153.

TABLE 1
Clinical characteristics of the patients with pancreatic
ductal adenocarcinoma and ampullary adenocarcinomas

| Characteristic | n = 225 |
|---|---|
| Age, years median (range) | 64 (31-85) |
| Sex, male/female | 111/114 |
| ASA-score, 1/2/3/4 | 2/174/49/0 |
| Localization of tumor, | 138/87 |
| PDAC/A-AC | |
| Stage, IA/IB/IIA/IIB * | 12/33/66/114 |
| Histological grade, | 69/58/81/16 |
| well/moderate/poor/undifferentiated ** | |
| KRAS, wild type/mutation | 58/167 |
| R0/R1 resections | 218/7 |
| Post operative chemotherapy+/− | 25/200 |

Values are numbers and percentages.
* Stage III and IV are inoperable patients.
** Un-reported for one patient.

TABLE 2
Hazard Ratios (HR) and confidence intervals (CI) for microRNAs associated with overall survival

| Unadjusted Estimates | | | | | Adjusted Estimates** | | | | |
|---|---|---|---|---|---|---|---|---|---|
| | | | Upper | | | | Lower | | |
| | | Lower | 95% | p- | | | 95% | Upper | |
| microRNA | HR* | 95% CI | CI | value | microRNA | HR* | CI | 95% CI | p-value |
| Overall survival | | | | | Overall survival | | | | |
| miR-212 | 1.54 | 1.21 | 1.96 | <0.001 | miR-212 | 1.32 | 1.02 | 1.72 | 0.033 |
| miR-675 | 1.08 | 1.02 | 1.14 | 0.009 | miR-675 | 1.06 | 1.00 | 1.14 | 0.051 |
| miR-148a* | 0.92 | 0.88 | 0.98 | 0.006 | miR-148a* | 0.94 | 0.88 | 1.00 | 0.039 |
| miR-187 | 0.97 | 0.94 | 1.00 | 0.061 | miR-187 | 0.96 | 0.93 | 0.99 | 0.010 |
| let-7g* | 0.83 | 0.73 | 0.95 | 0.006 | let-7g* | 0.78 | 0.68 | 0.90 | <0.001 |
| Two years follow-up*** | | | | | Two years follow-up*** | | | | |
| miR-675 | 1.13 | 1.06 | 1.22 | <0.001 | miR-675 | 1.14 | 1.05 | 1.34 | 0.001 |
| miR-148a* | 0.90 | 0.84 | 0.96 | 0.002 | miR-148a* | 0.88 | 0.82 | 0.95 | 0.002 |
| miR-146a | 0.62 | 0.48 | 0.79 | <0.001 | miR-146a | 0.51 | 0.39 | 0.68 | <0.001 |
| miR-450b- | 1.10 | 1.01 | 1.20 | 0.030 | miR-450b- | 1.14 | 1.04 | 1.30 | 0.001 |
| 5p | | | | | 5p | | | | |
| miR-222 | 1.34 | 1.032 | 1.75 | 0.026 | miR-222 | 1.39 | 1.06 | 1.84 | 0.004 |

*Calculated for per unit increase for the (40-Ct)-value.
**Adjusted for age, gender, calendar year of operation, KRAS mutation status, stage, ASA-score, localization of tumor and differentiation of tumor.
***All survival times above 24 months were censored at 24 months in the analysis.

TABLE 3
The microRNAs with strongest prediction ability of survival
by the Lasso method when sorted by size of effect

| Rank | Overall Survival | Rank | Two years follow-up* |
|---|---|---|---|
| 1 | miR-212 | 1 | miR-675 |
| 2 | miR-675 | 2 | miR-148a* |
| 3 | miR-187 | 3 | miR-205 |
| 4 | miR-205 | 4 | miR-23a* |
| 5 | miR-944 | 5 | miR-431 |
| 6 | miR-431 | 6 | miR-187 |
| 7 | miR-194* | 7 | miR-143* |
| 8 | miR-148a* | 8 | miR-216a |
| 9 | miR-769-5p | 9 | miR-450b-5p |
| | | 10 | miR-891a |
| | | 11 | miR-409-5p |
| | | 12 | miR-194* |
| | | 13 | miR-449b |
| | | 14 | miR-330-5p |

*All survival times above 24 months were censored in the analysis.

### ITEMS

1. A method for predicting the prognosis for a patient with pancreatic cancer, said method comprising measuring the expression level of at least one miRNA in a sample obtained from said individual, wherein the at least one miRNA is selected from the group consisting of
   a) miR-675, miR-212, miR-148a*, miR-187, let-7g*, miR-205, miR-944, miR-431, miR-194*, miR-769-5p, miR-450b-5p, miR-222, miR-146a, miR-23a*, miR-143*, miR-216a, miR-891a, miR-409-5p, miR-449b and miR-330-5p; or
   b) miR-675, miR-212, miR-148a*, miR-187 and let-7g*; or
   c) miR-675, miR-212, miR-148a*, miR-187, miR-205, miR-944, miR-431, miR-194* and miR-769-5p; or
   d) miR-675, miR-212, miR-148a* and miR-187; or
   e) miR-675, miR-148a*, miR-450b-5p, miR-222 and miR-146a; or
   f) miR-675, miR-148a*, miR-187, miR-205, miR-431, miR-194*, miR-23a*, miR-143*, miR-216a, miR-891a, miR-409-5p, miR-450b-5p, miR-449b and miR-330-5p;
   g) miR-675, miR-148a* and miR-450b-5p; or
   h) miR-675, miR-187, miR-205, miR-431, miR-194* and 148a*; or
   i) miR-675 and miR-148a*; or
   j) miR-675, miR222* and miR-29a*; or
   k) miR-148a and miR-625.
2. The method according to item 1, wherein said method is a method for estimating the probability for a patient with pancreatic cancer of surviving for a certain time period, wherein the miRNA expression level of at least one of said miRNAs is indicative of said individual with pancreatic carcinoma surviving for a certain time period.
3. The method according to item 1, said method further comprising the step of obtaining a sample from an individual with pancreas cancer.
4. The method according to item 1, said method further comprising the step of extracting RNA from a sample collected from an individual with pancreas cancer.
5. The method according to item 1, said method further comprising the step of correlating the miRNA expression level of at least one of said miRNAs to a predetermined reference level.
6. The method according to item 1, said method further comprising the step of determining whether or not said sample is indicative of the individual having a certain predicted prognosis.
7. The method according to item 1, said method further comprising the step of obtaining prediction probabilities of between 0-1 for said sample.
8. The method according to item 7, wherein said prediction probability is in the range of 0.01 to 0.1, such as 0.1 to 0.2, for example 0.2 to 0.3, such as 0.3 to 0.4, for example 0.4 to 0.5, such as 0.5 to 0.6, for example 0.6 to 0.7, such as 0.7 to 0.8, for example 0.8 to 0.85, such as 0.85 to 0.9, for example 0.9 to 0.91, such as 0.91 to 0.92, for example 0.92 to 0.93, such as 0.93 to 0.94, for example 0.94 to 0.95, such as 0.95 to 0.96, for example 0.96 to 0.97, such as 0.97 to 0.98, for example 0.98 to 0.99, such as 0.99 to 1.0.
9. The method according to item 2, wherein said time period is expressed as 3-months survival probability, 6-months survival probability, 9-months survival probability, 12-months survival/1-year survival probability, 2-years survival probability, 3-years survival probability, 4-years survival probability, 5-years survival probability, 6-years survival probability, 7-years survival probability, 8-years survival probability, 9-years survival probability or 10-years survival probability.
10. The method according to item 9, wherein said time period is calculated from time of diagnosis, time of surgery or time of analysis/evaluation.
11. The method according to item 1, wherein said sample obtained from an individual is a tissue sample.
12. The method according to item 11, wherein said tissue sample is a tissue sample from the pancreas.
13. The method according to item 12, wherein said pancreatic tissue sample comprises pancreatic carcinoma cells.
14. The method according to item 13, wherein said pancreatic tissue sample further comprises cells of the desmoplastic stroma surrounding the tumour, e.g. fibroblasts, pancreatic stellate cells, inflammatory cells (e.g. macrophages and neutrofils) and endothelial cells.
15. The method according to item 1, wherein said sample obtained from an individual is a blood sample.
16. The method according to item 1, wherein the expression level of at least one miRNA is altered as compared to the expression level in a reference sample.
17. The method according to item 16, wherein said reference sample is obtained from an individual with pancreas cancer having a known estimated prognosis.
18. The method according to item 1, wherein said pancreatic carcinoma is pancreatic adenocarcinoma.
19. The method according to item 1, wherein said pancreatic carcinoma is ampullary adenocarcinoma.
20. The method according to item 1, wherein said pancreatic carcinoma is pancreatic adenocarcinoma and/or ampullary adenocarcinoma.
21. The method according to item 1, wherein said at least one miRNA is selected from the group consisting of miR-675, miR-212, miR-148a*, miR-187, let-7g*, miR-205, miR-944, miR-431, miR-194*, miR-769-5p, miR-450b-5p, miR-222, miR-146a, miR-23a*, miR-143*, miR-216a, miR-891a, miR-409-5p, miR-449b and miR-330-5p.
22. The method according to item 1, wherein said at least one miRNA is selected from the group consisting of miR-675, miR-212, miR-148a*, miR-187 and let-7g*.
23. The method according to item 1, wherein said at least one miRNA is selected from the group consisting of miR-675, miR-212, miR-148a*, miR-187, miR-205, miR-944, miR-431, miR-194* and miR-769-5p.
24. The method according to item 1, wherein said at least one miRNA is selected from the group consisting of miR-675, miR-212, miR-148a* and miR-187.
25. The method according to item 1, wherein said at least one miRNA is selected from the group consisting of miR-675, miR-148a*, miR-450b-5p, miR-222 and miR-146a.
26. The method according to item 1, wherein said at least one miRNA is selected from the group consisting of miR-675, miR-148a*, miR-187, miR-205, miR-431, miR-194*, miR-23a*, miR-143*, miR-216a, miR-891a, miR-409-5p, miR-450b-5p, miR-449b and miR-330-5p.
27. The method according to item 1, wherein said at least one miRNA is selected from the group consisting of miR-675, miR-148a* and miR-450b-5p.
28. The method according to item 1, wherein said at least one miRNA is selected from the group consisting of miR-675, miR-187, miR-205, miR-431, miR-194* and 148a*.
29. The method according to item 1, wherein said at least one miRNA is selected from the group consisting of miR-675 and miR-148a*.
30. The method according to any of the preceding items, wherein said at least one miRNA comprises or consists of
   a) miR-675, miR-212, miR-148a*, miR-187, let-7g*, miR-205, miR-944, miR-431, miR-194*, miR-769-5p, miR-450b-5p, miR-222, miR-146a, miR-23a*, miR-143*, miR-216a, miR-891a, miR-409-5p, miR-449b and miR-330-5p; or
   b) miR-675, miR-212, miR-148a*, miR-187 and let-7g*; or
   c) miR-675, miR-212, miR-148a*, miR-187, miR-205, miR-944, miR-431, miR-194* and miR-769-5p; or
   d) miR-675, miR-212, miR-148a* and miR-187; or
   e) miR-675, miR-148a*, miR-450b-5p, miR-222 and miR-146a; or
   f) miR-675, miR-148a*, miR-187, miR-205, miR-431, miR-194*, miR-23a*, miR-143*, miR-216a, miR-891a, miR-409-5p, miR-450b-5p, miR-449b and miR-330-5p;
   g) miR-675, miR-148a* and miR-450b-5p; or
   h) miR-675, miR-187, miR-205, miR-431, miR-194* and 148a*; or
   i) miR-675 and miR-148a*;
   j) miR-675, miR222* and miR-29a*; or
   k) miR-148a and miR-625.
31. The method according to item 1, wherein the expression level of 2 miRNAs is measured.
32. The method according to item 1, wherein the expression level of at least 2 miRNA is measured, such as 2 miRNAs, such as 3 miRNAs, for example 4 miRNAs, such as 5 miRNAs, for example 6 miRNAs, such as 7 miRNAs, for example 8 miRNAs, such as 9 miRNAs, for example 10 miRNAs, such as 11 miRNAs, for example 12 miRNAs, such as 13 miRNAs, for example 14 miRNAs, such as 15 miRNAs, for example 16 miRNAs, such as 17 miRNAs, for example 18 miRNAs, such as 19 miRNAs, for example 20 miRNAs.
33. The method according to item 1, wherein the expression level of one or more additional miRNAs is measured, said one or more miRNA having previously been identified in the literature as being of prognostic value.
34. The method according to item 33, wherein said additional miRNA comprise 1 additional miRNA, for example 2 additional miRNAs, such as 3 additional miRNA, for example 4 additional miRNAs, such as 5 additional miRNA, for example 6 additional miRNAs, such as 7 additional miRNA, for example 8 additional miRNAs, such as 9 additional miRNA, for example 10 additional miRNAs, such as 11 additional miRNA, for example 12 additional miRNAs, such as 13 additional miRNA, for example 14 additional miRNAs, such as 15 additional miRNAs, for example 16 additional miRNAs, such as 17 additional miRNA, for example 18 additional miRNAs, such as 19 additional miRNAs, for example 20 additional miRNAs.
35. The method according to item 1, wherein said method is used in combination with at least one additional prognostic method.
36. The method according to item 1, wherein the expression level of the at least one miRNA is determined by the microarray technique.
37. The method according to item 1, wherein the expression level of the at least one miRNA is determined by the quantitative polymerase chain reaction (QPCR) technique.
38. The method according to item 1, wherein the expression level of the at least one miRNA is determined by the northern blot technique.
39. The method according to item 1, wherein the expression level of the at least one miRNA is determined by Nuclease protection assay.
40. The method according to item 1, wherein the sample is extracted from an individual by fine-needle aspiration.
41. The method according to item 1, wherein the sample is extracted from an individual by coarse-needle aspiration.
42. The method according to item 1, wherein the sample is extracted from an individual by pancreatic surgery.
43. The method according to item 1, wherein the sample is extracted from an individual by pancreatic biopsy.
44. A device for measuring the expression level of at least one miRNA in a sample, wherein said device comprises or consists of at least one probe or probe set for at least one miRNA selected from the group consisting of
   a) miR-675, miR-212, miR-148a*, miR-187, let-7g*, miR-205, miR-944, miR-431, miR-194*, miR-769-5p, miR-450b-5p, miR-222, miR-146a, miR-23a*, miR-143*, miR-216a, miR-891a, miR-409-5p, miR-449b and miR-330-5p; or
   b) miR-675, miR-212, miR-148a*, miR-187 and let-7g*; or
   c) miR-675, miR-212, miR-148a*, miR-187, miR-205, miR-944, miR-431, miR-194* and miR-769-5p; or
   d) miR-675, miR-212, miR-148a* and miR-187; or
   e) miR-675, miR-148a*, miR-450b-5p, miR-222 and miR-146a; or
   f) miR-675, miR-148a*, miR-187, miR-205, miR-431, miR-194*, miR-23a*, miR-143*, miR-216a, miR-891a, miR-409-5p, miR-450b-5p, miR-449b and miR-330-5p;
   g) miR-675, miR-148a* and miR-450b-5p; or
   h) miR-675, miR-187, miR-205, miR-431, miR-194* and 148a*; or
   i) miR-675 and miR-148a*; or
   j) miR-675, miR222* and miR-29a*; or
   k) miR-148a and miR-625,
   wherein said device is used for characterising a sample.
45. The device according to item 44, wherein said device further comprises one or more probes or probe sets for one or more miRNA having previously been identified in the literature as being of prognostic value.
46. The device according to item 44, wherein said device is used in a method for estimating the probability for a patient with pancreatic cancer of surviving for a certain time period, said method comprising measuring the expression level of at least one miRNA in a sample obtained from said individual.
47. The device according to item 44, wherein said device comprises or consists of a total of 1 probe or probe set, such as 2 probes, for example 3 probes, such as 4 probes, for example 5 probes, such as 6 probes, for example 7 probes, such as 8 probes, for example 9 probes, such as 10 probes, for example 11 probes, such as 12 probes, for example 13 probes, such as 14 probes, for example 15 probes, such as 16 probes, for example 17 probes, such as 18 probes, for example 19 probes, such as 20 probes, for example 21 probes, such as 22 probes, for example 23 probes, such as 24 probes, for example 25 probes, such as 26 probes, for example 27 probes, such as 28 probes, for example 29 probes, such as 30 probes, for example 31 probes, such as 32 probes, for example 33 probes, such as 34 probes, for example 35 probes, such as 36 probes, for example 37 probes, such as 38 probes, for example 39 probes, such as 40 probes, for example 41 probes, such as 42 probes, for example 43 probes, such as 44 probes, for example 45 probes, such as 46 probes, for example 47 probes, such as 48 probes, for example 49 probes, such as 50 probes or probe sets.
48. The device according to item 44, wherein said device is a microarray chip.
49. The device according to item 48, wherein said device is a microarray chip comprising DNA probes.
50. The device according to item 48, wherein said device is a microarray chip comprising antisense miRNA probes.
51. The device according to item 44, wherein said device is a QPCR Microfluidic Card.
52. The device according to item 44, wherein said device comprises QPCR tubes, QPCR tubes in a strip or a QPCR plate.
53. The device according to item 44, wherein said device comprises probes on a solid support.
54. The device according to item 44, wherein said device comprises probes on at least one bead.
55. The device according to item 44, wherein said device comprises probes in liquid form in a tube.
56. A kit-of-parts comprising the device of item 44, and at least one additional component.
57. The kit according to item 56, wherein said additional component comprises means for extracting RNA, such as miRNA, from a sample.
58. The kit according to item 56, wherein said additional component comprises reagents for performing microarray analysis.
59. The kit according to item 56, wherein said additional component comprises reagents for performing QPCR analysis.
60. The kit according to item 56, wherein said additional component is the computer program product according to item 64.
61. The kit according to item 56, wherein said additional component is instructions for use of the device.
62. A system for predicting the prognosis for a patient with pancreatic cancer, comprising means for analysing the expression level of at least one miRNA in a sample obtained from an individual with pancreas cancer, wherein said at least one miRNA is selected from the group consisting of
   a) miR-675, miR-212, miR-148a*, miR-187, let-7g*, miR-205, miR-944, miR-431, miR-194*, miR-769-5p, miR-450b-5p, miR-222, miR-146a, miR-23a*, miR-143*, miR-216a, miR-891a, miR-409-5p, miR-449b and miR-330-5p; or
   b) miR-675, miR-212, miR-148a*, miR-187 and let-7g*; or
   c) miR-675, miR-212, miR-148a*, miR-187, miR-205, miR-944, miR-431, miR-194* and miR-769-5p; or
   d) miR-675, miR-212, miR-148a* and miR-187; or
   e) miR-675, miR-148a*, miR-450b-5p, miR-222 and miR-146a; or
   f) miR-675, miR-148a*, miR-187, miR-205, miR-431, miR-194*, miR-23a*, miR-143*, miR-216a, miR-891a, miR-409-5p, miR-450b-5p, miR-449b and miR-330-5p;
   g) miR-675, miR-148a* and miR-450b-5p; or
   h) miR-675, miR-187, miR-205, miR-431, miR-194* and 148a*; or
   i) miR-675 and miR-148a*; or
   j) miR-675, miR222* and miR-29a*; or
   k) miR-148a and miR-625.
63. A system for predicting the prognosis on an individual with pancreas cancer, comprising:
   i) means for analysing the miRNA expression profile of a sample obtained from said individual, and
   ii) means for estimating the probability for a patient with pancreatic cancer of surviving for a certain time period,
   wherein said miRNA expression profile comprises at least one miRNA selected from the group consisting of
   a) miR-675, miR-212, miR-148a*, miR-187, let-7g*, miR-205, miR-944, miR-431, miR-194*, miR-769-5p, miR-450b-5p, miR-222, miR-146a, miR-23a*, miR-143*, miR-216a, miR-891a, miR-409-5p, miR-449b and miR-330-5p; or
   b) miR-675, miR-212, miR-148a*, miR-187 and let-7g*; or
   c) miR-675, miR-212, miR-148a*, miR-187, miR-205, miR-944, miR-431, miR-194* and miR-769-5p; or
   d) miR-675, miR-212, miR-148a* and miR-187; or
   e) miR-675, miR-148a*, miR-450b-5p, miR-222 and miR-146a; or
   f) miR-675, miR-148a*, miR-187, miR-205, miR-431, miR-194*, miR-23a*, miR-143*, miR-216a, miR-891a, miR-409-5p, miR-450b-5p, miR-449b and miR-330-5p;
   g) miR-675, miR-148a* and miR-450b-5p; or
   h) miR-675, miR-187, miR-205, miR-431, miR-194* and 148a*; or
   i) miR-675 and miR-148a*; or
   j) miR-675, miR222* and miR-29a*; or
   k) miR-148a and miR-625.
64. A computer program product having a computer readable medium, said computer program product providing a system for predicting the prognosis of an individual, said computer program product comprising means for carrying out any of the steps of any of the methods according to any of items 62 to 63.
65. A system according to any of items 62 to 63, wherein the data is stored, such as stored in at least one database.
66. The method according to claim 1, wherein said patient is diagnosed with pancreatic cancer of the type PDAC, said method comprising measuring the expression level of at least one miRNA selected from the group consisting of miR-675, miR222* and miR-29a*.
67. The method according to claim 1, wherein said patient is diagnosed with pancreatic cancer of the type A-AC, said method comprising measuring the expression level of at least one miRNA selected from the group consisting of miR-148a and miR-625.

1. A method for predicting the prognosis for a patient with pancreatic cancer, said method comprising measuring the expression level of at least one miRNA in a sample obtained from said individual, wherein the at least one miRNA is selected from the group consisting of
a) miR-675, miR-212, miR-148a*, miR-187 and let-7g*; or
b) miR-675, miR-212, miR-148a* and miR-187; or
c) miR-675, miR-212, miR-148a* and let-7g*; or
d) miR-675, miR-212, miR-148a*, miR-187, miR-205, miR-944, miR-431, miR-194* and miR-769-5p; or
e) miR-675, miR-212, miR-148a*, miR-187, let-7g*, miR-205, miR-944, miR-431, miR-194*, miR-769-5p, miR-450b-5p, miR-222, miR-146a, miR-23a*, miR-143*, miR-216a, miR-891a, miR-409-5p, miR-449b and miR-330-5p; or
f) miR-675, miR-148a*, miR-450b-5p, miR-222 and miR-146a; or
g) miR-675, miR-148a*, miR-187, miR-205, miR-431, miR-194*, miR-23a*, miR-143*, miR-216a, miR-891a, miR-409-5p, miR-450b-5p, miR-449b and miR-330-5p;
h) miR-675, miR-148a* and miR-450b-5p; or
i) miR-675, miR-187, miR-205, miR-431, miR-194* and 148a*; or
j) miR-675 and miR-148a*, or
k) miR-675, miR222*, miR-29a*, miR-148a and miR-625.
wherein said method is a method for estimating the probability for a patient with pancreatic cancer of surviving for a certain time period, wherein the miRNA expression level of at least one of said miRNAs is indicative of said individual with pancreatic carcinoma surviving for a certain time period.

2. The method according to claim 1, said method further comprising the step of obtaining a sample from an individual with pancreas cancer and/or extracting RNA from a sample collected from an individual with pancreas cancer.

3. The method according to claim 1, said method further comprising the step of correlating the miRNA expression level of at least one of said miRNAs to a predetermined reference level.

4. The method according to claim 1, said method further comprising the step of determining whether or not said sample is indicative of the individual having a certain predicted prognosis.

5. The method according to claim 1, said method further comprising the step of obtaining prediction probabilities of between 0-1 for said sample.

6. The method according to claim 1, wherein said time period is expressed as 3-months survival probability, 6-months survival probability, 9-months survival probability, 12-months survival/1-year survival probability, 2-years survival probability, 3-years survival probability, 4-years survival probability, 5-years survival probability, 6-years survival probability, 7-years survival probability, 8-years survival probability, 9-years survival probability or 10-years survival probability.

7. The method according to claim 1, wherein said sample obtained from an individual is a tissue sample, such as a tissue sample from the pancreas, such as a tissue sample comprising pancreatic carcinoma cells.

8. The method according to claim 1, wherein said sample obtained from an individual is a blood sample.

9. The method according to claim 1, wherein said pancreatic carcinoma is pancreatic adenocarcinoma and/or ampullary adenocarcinoma.

10. The method according to any of the preceding claims, comprising measuring the expression level of a group of miRNAs in a sample obtained from said individual, said group consisting of
a) miR-675, miR-212, miR-148a*, miR-187 and let-7g*; or
b) miR-675, miR-212, miR-148a* and miR-187; or
c) miR-675, miR-212, miR-148a* and let-7g*; or
d) miR-675, miR-212, miR-148a*, miR-187, miR-205, miR-944, miR-431, miR-194* and miR-769-5p; or
e) miR-675, miR-212, miR-148a*, miR-187, let-7g*, miR-205, miR-944, miR-431, miR-194*, miR-769-5p, miR-450b-5p, miR-222, miR-146a, miR-23a*, miR-143*, miR-216a, miR-891a, miR-409-5p, miR-449b and miR-330-5p; or
f) miR-675, miR-148a*, miR-450b-5p, miR-222 and miR-146a; or
g) miR-675, miR-148a*, miR-187, miR-205, miR-431, miR-194*, miR-23a*, miR-143*, miR-216a, miR-891a, miR-409-5p, miR-450b-5p, miR-449b and miR-330-5p;
h) miR-675, miR-148a* and miR-450b-5p; or
i) miR-675, miR-187, miR-205, miR-431, miR-194* and 148a*; or
j) miR-675 and miR-148a*; or
k) miR-675, miR222*, miR-29a*, miR-148a and miR-625.

11. The method according to any of the preceding claims, wherein the expression level of 2 miRNA is measured, such as 3 miRNAs, for example 4 miRNAs, such as 5 miRNAs, for example 6 miRNAs, such as 7 miRNAs, for example 8 miRNAs, such as 9 miRNAs, for example 10 miRNAs, such as 11 miRNAs, for example 12 miRNAs, such as 13 miRNAs, for example 14 miRNAs, such as 15 miRNAs, for example 16 miRNAs, such as 17 miRNAs, for example 18 miRNAs, such as 19 miRNAs, for example 20 miRNAs is measured.

12. The method according to claim 1, wherein the expression level of one or more additional miRNAs is measured, said one or more additional miRNA having previously been identified in the literature as being of prognostic value.

13. The method according to claim 1, wherein said method is used in combination with at least one additional prognostic method.

14. The method according to claim 1, wherein the expression level of the at least one miRNA is determined by the microarray technique, or the quantitative polymerase chain reaction (QPCR) technique, or the northern blot technique, or the nuclease protection assay technique.

15. The method according to claim 1, wherein the sample is extracted from an individual by fine-needle aspiration, or coarse-needle aspiration, or pancreatic surgery, or a pancreatic biopsy.

16. A device for measuring the expression level of at least one miRNA in a sample, wherein said device comprises or consists of at least one probe or probe set for at least one miRNA selected from the group consisting of
a) miR-675, miR-212, miR-148a*, miR-187 and let-7g*; or
b) miR-675, miR-212, miR-148a* and miR-187; or
c) miR-675, miR-212, miR-148a* and let-7g*; or
d) miR-675, miR-212, miR-148a*, miR-187, miR-205, miR-944, miR-431, miR-194* and miR-769-5p; or
e) miR-675, miR-212, miR-148a*, miR-187, let-7g*, miR-205, miR-944, miR-431, miR-194*, miR-769-5p, miR-450b-5p, miR-222, miR-146a, miR-23a*, miR-143*, miR-216a, miR-891a, miR-409-5p, miR-449b and miR-330-5p; or
f) miR-675, miR-148a*, miR-450b-5p, miR-222 and miR-146a; or
g) miR-675, miR-148a*, miR-187, miR-205, miR-431, miR-194*, miR-23a*, miR-143*, miR-216a, miR-891a, miR-409-5p, miR-450b-5p, miR-449b and miR-330-5p;
h) miR-675, miR-148a* and miR-450b-5p; or
i) miR-675, miR-187, miR-205, miR-431, miR-194* and 148a*; or
j) miR-675 and miR-148a*, or
k) miR-675, miR222*, miR-29a*, miR-148a and miR-625,
wherein said device is used for characterising a sample obtained from a patient with pancreatic cancer.

17. The device according to claim 11, wherein said device further comprises one or more probes or probe sets for one or more miRNA having previously been identified in the literature as being of prognostic value.

18. The device according to claims 11-12, wherein said device is used in a method for estimating the probability for a patient with pancreatic cancer of surviving for a certain time period, said method comprising measuring the expression level of at least one miRNA in a sample obtained from said individual.

19. The device according to claim 11, wherein said device is a microarray chip, a QPCR Microfluidic Card, QPCR tubes or a QPCR plate.

20. A kit-of-parts comprising the device of claim 11 and at least one additional component.

21. The kit according to claim 20, wherein said additional component comprises means for extracting RNA such as miRNA, from a sample; reagents for performing microarray analysis; reagents for performing QPCR analysis; a computer program product according to claim 23 and/or instructions for use of the device.

22. A system for predicting the prognosis on an individual with pancreas cancer, comprising:
i) means for analysing the miRNA expression profile of a sample obtained from said individual, and
ii) means for estimating the probability for a patient with pancreatic cancer of surviving for a certain time period,
wherein said miRNA expression profile comprises at least one miRNA selected from the group consisting of
a) miR-675, miR-212, miR-148a*, miR-187 and let-7g*; or
b) miR-675, miR-212, miR-148a* and miR-187; or
c) miR-675, miR-212, miR-148a* and let-7g*; or
d) miR-675, miR-212, miR-148a*, miR-187, miR-205, miR-944, miR-431, miR-194* and miR-769-5p; or
e) miR-675, miR-212, miR-148a*, miR-187, let-7g*, miR-205, miR-944, miR-431, miR-194*, miR-769-5p, miR-450b-5p, miR-222, miR-146a, miR-23a*, miR-143*, miR-216a, miR-891a, miR-409-5p, miR-449b and miR-330-5p; or
f) miR-675, miR-148a*, miR-450b-5p, miR-222 and miR-146a; or
g) miR-675, miR-148a*, miR-187, miR-205, miR-431, miR-194*, miR-23a*, miR-143*, miR-216a, miR-891a, miR-409-5p, miR-450b-5p, miR-449b and miR-330-5p;
h) miR-675, miR-148a* and miR-450b-5p; or
i) miR-675, miR-187, miR-205, miR-431, miR-194* and 148a*; or
j) miR-675 and miR-148a*, or
k) miR-675, miR222*, miR-29a*, miR-148a and miR-625

23. A computer program product having a computer readable medium, said computer program product providing a system for predicting the prognosis of an individual, said computer program product comprising means for carrying out any of the steps of any of the methods according to claim 22.


SEQUENCE LISTING (24 sequences)

SEQ ID NO: 1 - RNA, 21 nt, Homo sapiens
cuguacaggccacugccuugc

SEQ ID NO: 2 - RNA, 23 nt, Homo sapiens
uggugcggagagggcccacagug

SEQ ID NO: 3 - RNA, 21 nt, Homo sapiens
uaacagucuccagucacggcc

SEQ ID NO: 4 - RNA, 22 nt, Homo sapiens
aaaguucugagacacuccgacu

SEQ ID NO: 5 - RNA, 22 nt, Homo sapiens
ucgugucuuguguugcagccgg

SEQ ID NO: 6 - RNA, 22 nt, Homo sapiens
uccuucauuccaccggagucug

SEQ ID NO: 7 - RNA, 22 nt, Homo sapiens
aaauuauuguacaucggaugag

SEQ ID NO: 8 - RNA, 21 nt, Homo sapiens
ugucuugcaggccgucaugca

SEQ ID NO: 9 - RNA, 22 nt, Homo sapiens
ccaguggggcugcuguuaucug

SEQ ID NO: 10 - RNA, 22 nt, Homo sapiens
ugagaccucuggguucugagcu

SEQ ID NO: 11 - RNA, 22 nt, Homo sapiens
uuuugcaauauguuccugaaua

SEQ ID NO: 12 - RNA, 21 nt, Homo sapiens
agcuacaucuggcuacugggu

SEQ ID NO: 13 - RNA, 22 nt, Homo sapiens
ugagaacugaauuccauggguu

SEQ ID NO: 14 - RNA, 22 nt, Homo sapiens
gggguuccuggggaugggauuu

SEQ ID NO: 15 - RNA, 22 nt, Homo sapiens
ggugcagugcugcaucucuggu

SEQ ID NO: 16 - RNA, 22 nt, Homo sapiens
uaaucucagcuggcaacuguga

SEQ ID NO: 17 - RNA, 22 nt, Homo sapiens
ugcaacgaaccugagccacuga

SEQ ID NO: 18 - RNA, 23 nt, Homo sapiens
agguuacccgagcaacuuugcau

SEQ ID NO: 19 - RNA, 22 nt, Homo sapiens
aggcaguguauuguuagcuggc

SEQ ID NO: 20 - RNA, 22 nt, Homo sapiens
ucucugggccugugucuuaggc

SEQ ID NO: 21 - RNA, 22 nt, Homo sapiens
cucaguagccaguguagauccu

SEQ ID NO: 22 - RNA, 22 nt, Homo sapiens
acugauuucuuuugguguucag

SEQ ID NO: 23 - RNA, 22 nt, Homo sapiens
ucagugcacuacagaacuuugu

SEQ ID NO: 24 - RNA, 22 nt, Homo sapiens
gacuauagaacuuucccccuca